(19) United States
(12) Patent Application Publication
(10) Pub. No.: US 2019/0316211 A1
(43) Pub. Date: Oct. 17, 2019

(54) ONCOGENIC ALTERNATIVE SPLICING SWITCH OF PACE4 IN CANCER

(71) Applicant: SOCIETE DE COMMERCIALISATION DES PRODUITS DE LA RECHERCHE APPLIQUEE SOCPRA SCIENCES SANTE ET HUMAINE, Sherbrooke (CA)

(72) Inventors: Robert DAY, Sherbrooke (CA); Frédéric COUTURE, Sherbrooke (CA); Robert SABBAGH, Sherbrooke (CA)

(73) Assignee: SOCIETE DE COMMERCIALISATION DES PRODUITS DE LA RECHERCHE APPLIQUEE SOCPRA SCIENCES SANTE ET HUMAINE, Sherbrooke, QC (CA)

(21) Appl. No.: 16/463,476

(22) PCT Filed: Nov. 29, 2017
(86) PCT No.: PCT/CA2017/051431

§ 371 Date: May 23, 2019

Related U.S. Application Data
(60) Provisional application No. 62/565,276, filed on Sep. 29, 2017.
(60) Provisional application No. 62/427,209, filed on Nov. 29, 2016.

(51) Int. Cl. C12Q 1/6886; C07K 16/40; A61K 38/43; G01N 33/574

(52) U.S. Cl. CPC C12Q 1/6886; C07K 16/40; G01N 2333/96438; G01N 33/57434; C12Q 2600/156; A61K 38/43

(57) ABSTRACT

It is provided a tumor promoting alternative splicing isoform of PACE4, named PACE4-alt CT, analytes specifically binding to PACE4-alt CT, such as antibodies, and use of same for detecting a cancer, such as prostate cancer. It is also provided a method treating a cancer in a patient comprising administering an inhibitor of PACE4-altCT to a patient in need thereof and a kit comprising an analyte specific reagent specifically binding to PACE4-altCT for detecting a cancer.

### CROSS-REFERENCE TO RELATED APPLICATIONS

The present application claims benefit of U.S. Provisional Application No. 62/427,209 filed Nov. 29, 2016, U.S. Provisional Application No. 62/565,276 filed Sep. 29, 2017, the content of which are hereby incorporated by reference in their entirety.

### TECHNICAL FIELD

It is provided an alternative splicing (PACE4-altCT) for PACE4 and use of same for detecting and/or treating cancer.

### BACKGROUND

Among malignancies, prostate cancer (PCa) remains the most common type of cancer in men with 233,000 cases each year in the USA; representing 27% of all new cases, as well as the second cause of cancer-related mortality. When diagnosed in its early progression stages, clinical interventions are able to circumvent disease progression and yield high survival rates over 5-15 years. However, when tumor initiated metastatic dissemination at the time of diagnostic or following tumor relapse, survival rates drop considerably, leading to patient death within 5 years in about 75% of cases. Yet, there is virtually no markers allowing the discrimination between cancer that will remain indolent from the high-risk malignancies. Moreover, most antineoplastic agents used for the management of advanced PCa are restricted to traditional chemotherapies and androgen axis manipulating agents. Various molecular targets have been envisaged for therapeutics which have not yielded sufficient survival gain when tested on patients, including angiogenic factors (anti-VEGF/VEGFR), tyrosine kinase receptor and downstream growth-promoting pathways, growth factors and active proteases, such as metalloproteinases (MMPs) or A disintegrin and metalloproteinases (ADAMs). Novel therapeutic avenues arising from yet unexplored biological pathways could provide a solution, either alone or as co-targets.

Among potential targets that have yet to be fully defined are the pro-protein convertases (PCs). These enzymes are responsible for the posttranslational processing of pro-protein substrates and are composed of nine members, namely; furin, PACE4, PC5/6, PC7, PC1/3, PC2, PC4, PCSK9 and SKI-1. The first seven are calcium-dependent serine proteases cleaving at paired basic residues with the consensus cleavage site R-X-(K/R)-R←. The PCs have been loosely associated with malignancies because of their capabilities to enhance the activity of cancer-associated protein substrates, which are overexpressed by tumor cells, e.g. members of the ADAM family of proteases, transforming growth factor-β (TGF-β), MMPs and IGF1R family members.

Among the PCs, PACE4 has been associated with malignant transformation in cancer cell based assays. However, in humans, a clear association of PACE4 overexpression and cancer has been, wherein a PACE4 overexpression in PCa tumours was observed (D'Anjou et al., 2011, Translational Oncology, 4: 157-172) while other PCs levels were not significantly altered. Subsequent studies using animal models supported the hypothesis that PACE4 overexpression has a role in PCa tumour progression, since molecular silencing of PACE4 in PCa xenograft animal models inhibited tumour growth, while the molecular silencing of other PCs did not (Couture et al., 2012, Neoplasia, 14: 1032-1042). These observations led to the development of PACE4-inhibitors (WO2010/003231 and WO 2013/029180) that mimicked molecular silencing, displaying anti-tumoral properties in xenograft models of PCa with increased cell quiescence and reduction of tumor neovascularization (Levesque et al., 2015, Oncotarget, 6: 3680-3693). In spite of this remarkable advancement and in vivo proof of concept, still little is known concerning the mechanisms associated with the sustained PACE4 overexpression in PCa cells and questions remain concerning the possibility that PACE4 levels have a relationship with disease outcome e.g., with tumor aggressiveness and/or patient survival. Various reports have highlighted the spatial and temporal regulation of PACE4 expression and its role in the regulation of embryonic developmental stages. However, there is relatively little information on the regulation of PACE4 expression in a pathophysiologic context despite the fluctuating levels across tissues, in various types of cancers, in osteoarthritis and atherosclerosis. No PACE4-specific substrates have been identified in cancer cells, leaving unexplained the identity of the substrates involved in the observed phenotype of PACE4 knockdown cancer cells. Thus the positioning of PACE4 as a therapeutic target, or as a potential biomarker, in the continuum of PCa disease cannot be fully understood, other than stating that the observed pharmacological effects are the resultant of a wide spectrum of downstream factors.

PCa cells are known to rapidly adapt to anti-androgen therapies and thus counter their anti-proliferation effects as they become androgen-independent. In spite of this, most therapies for PCa management used today have an androgen-based mechanism of action. In contrast to this unchanged continuum, researchers have attempted to define extra-androgenic pathways such as the promiscuous growth factor pathways which are used to either substitute androgen-receptor ligand requirement to activate the receptor (also known as the outlaw pathways) or to directly regulate key cancer cell capabilities such as proliferation, angiogenesis, immunosuppression through their action on either oncogenes or tumor suppressor gene pathways. In normal prostate, growth factors are secreted to act as paracrine and autocrine fine-tuning agents in the regulation of prostatic growth and differentiation. However, when PCa cells emerge, and more importantly when the disease progresses from early to late stages, several alterations in growth factors and their receptors as well as pro-invasive matrix modifying enzymes leads to a drastic changes from paracrine to autocrine mediation of sustained proliferation. Whether these alterations are causal or collateral to oncogenic transformation is often hard to define knowing the strong heterogeneity of PCa.

It is thus highly desired to be provided with novel molecular targets for cancer therapeutics.

### SUMMARY

In accordance with the present disclosure there is provided a method for detecting a cancer in a subject comprising the steps of obtaining a biological sample from the subject; and detecting the cancer by detecting the presence of PACE4-altCT in the biological sample.

In accordance with the present disclosure there is also provided an antibody specifically binding to PACE4-altCT.

It is further provided a kit comprising an analyte specific reagent specifically binding to PACE4-altCT; and instruction for use.

In an embodiment, the method described herein comprises contacting an analyte specific reagent specifically binding to PACE4-altCT with the biological sample under conditions so as to allow the formation of an analyte-PACE4-altCT complex; and detecting the cancer by detecting the analyte-PACE4-altCT complex.

In another embodiment, the method described herein further comprises the step of detecting the expression of GDF-15 prior or after the detection of the presence of PACE4-altCT in the biological sample.

In an embodiment, the method described herein further comprises the step of detecting the presence of PACE4-FL in the biological sample and calculating a ratio of PACE4-altCT/PACE4-FL wherein a ration of above 2 is indicative of the presence of the cancer.

In an additional embodiment the sample is a blood sample, urine, a tissue specimen, a biopsy needle washes, or circulating cells.

In another embodiment, the analyte is an antibody, a peptide, a primer or a probe.

In another embodiment, the antibody is a monoclonal antibody, a humanized antibody or a polyclonal antibody.

In a further embodiment, the antibody is a mouse antibody, a goat antibody, a human antibody, chicken, donkey, camelid, alpaga, turkey or a rabbit antibody.

In an additional embodiment, the antibody specifically binds to SEQ ID NO: 9.

In another embodiment, the antibody specifically binds to an epitope comprising the amino acid sequence set forth in any one of SEQ ID NOs: 18, 23, 24, 25, and 26.

In a further embodiment, the PACE4-altCT detected is a protein or a nucleic acid molecule.

In an embodiment, the nucleic acid molecule is an RNA or a DNA molecule.

In an embodiment, the probe is an oligonucleotide or a siRNA molecule.

In another embodiment, the probe specifically binds to a nucleotide sequence comprising SEQ ID NOs: 4, 5 or 6.

In a further embodiment, the siRNA comprises the nucleotide sequence set forth in SEQ ID NOs: 10, 11, 12, 13, 14, 15 or 16.

In another embodiment, the method described herein further comprises the step of applying a detection agent that detects the analyte-PACE4altCT complex.

In an embodiment, the detection agent is detected by Western blot, ELISA, immunoprecipitation followed by SDS-PAGE, immunocytochemistry, immunohistochemistry, PCR, or RT-PCR.

In a further embodiment, the PACE4-altCT is detected by mass spectrometry.

In an additional embodiment, the PACE4-altCT is detected by LC-MS/MS quantification.

In a further embodiment, the cancer is in at least one of lungs, thyroid, adrenals, testis, endometrium, pancreas, oesophagus, prostate, ovary, liver, breast, colon, stomach, kidney, bladder, brain, cervix, and lymphoid tissues.

In a specific embodiment, the cancer is a prostate cancer.

In an embodiment, the kit escribed herein further comprises an analyte specific reagent specifically binding to PACE4-FL.

In another embodiment, the kit escribed herein further comprises a detection agent that detects the analyte.

It is further provided the use of an analyte specific reagent specifically binding to PACE4-altCT for detecting a cancer in a sample of a subject.

It is also provided a method of treating a cancer in a patient comprising administering an inhibitor of PACE4-altCT to a patient in need thereof.

In an embodiment, the inhibitor is a siRNA, an antibody or a peptide.

In another embodiment, the peptide comprises the following formula:

Y-Arg₄-Xaa₃-Xaa₂-Arg₁-NH₂;
wherein
Arg₁ is an arginine, or an arginine derivative;
Xaa₂ and Xaa₃ are any amino acids or stereoisomers thereof; and
Y is absent or comprises the formula Z-Xaa₈-Xaa₇-Xaa₆-Xaa₅, wherein
   Xaa₅, Xaa₆, Xaa₇ and Xaa₈ are independently selected from the group consisting of Lys, His and Arg;
   Z is absent or comprises an N-terminal acyl group linked to the N-terminal of the peptide sequence;
   with the proviso that Xaa₅, Xaa₆, Xaa₇ and Xaa₈ are not aromatic or negatively charged amino acids.

In a further embodiment, Xaa₅, Xaa₆, Xaa₇ and Xaa₈ are positively charged amino acids or stereoisomers thereof.

In another embodiment, Xaa₃ is Val.

In a supplemental embodiment, wherein Xaa₂ and Xaa₃ are independently selected from Gly and Ala.

In an embodiment, Xaa₂ is Lys or Arg.

In another embodiment, Xaa₅, Xaa₆, Xaa₇ and Xaa₈ are aliphatic hydrophobic amino acids.

In another embodiment, the aliphatic hydrophobic amino acids are Leu, Iso or Val.

In an additional embodiment, Xaa₅, Xaa₆, Xaa₇ and Xaa₈ are Leu.

In another embodiment, the peptide consists of Ac-LLLLRVK-[AMBA]; Ac-[D-Leu]-LLLRVK-[AMBA]; Ac-LLLIRVK-[AMBA]; Ac-[D-Leu]-LLIRVK-[AMBA]; Ac-LLILRVK-[AMBA]; Ac-[D-Leu]-LILRVK-[AMBA]; Ac-LLLQRVK-[AMBA]; Ac-[D-Leu]-LLQRVK-[AMBA]; Ac-[Azaβ₃L]LLLRVK-[ΔR-COO]; Ac-LLLLRVK-[ΔR-COO]; Ac-[D-Leu]-LLLRVK-[ΔR-CO].

It is provided the use of an inhibitor of PACE4-altCT for treating a cancer in a patient.

It is also provided a method of treating a cancer in a patient comprising administering an inhibitor of PACE4-altCT to a patient in need thereof.

It is further provided a composition for treating cancer comprising an inhibitor of PACE4-altCT and a carrier.

In an embodiment, the inhibitor of PACE4-altCT is an antibody or an siRNA.

In an embodiment, the siRNA is complementary to a sequence selected from the group consisting of: SEQ ID NO: 4, 5 and 6.

In another embodiment, the siRNA comprises the nucleotide sequence set forth in SEQ ID NOs: 10, 11, 12, 13, 14, 15 or 16.

### BRIEF DESCRIPTION OF THE DRAWINGS

Reference will now be made to the accompanying drawings.

FIG. 1 illustrates that PACE4 expression correlates with prostate cancer tumor aggressiveness, wherein in (A) qPCR analysis of PACE4 expression levels in fresh prostate tissues specimens showing a significant correlation between levels and tumor Gleason scores; (B) similar analysis retrieved using c-BioPortal for Cancer Genomics using the Broad/Cornel Nature Genetics 2012 and MSKCC Cancer Cell 2010; (C to H) PACE4 IHC (using catalytic domain targeting antibody) in PCa representative specimens showing concomitant enhancement of PACE4 expression at the protein level, wherein r values are Spearman's correlation coefficients and scale bars represent 200 μm.

FIG. 2 illustrates alternative splicing of PACE4 terminal exon results in 3′UTR shortening and stabilize mRNA transcripts, showing in (A) LNCaP 3′RACE PCR products primed on exon 23 runned on agarose gel (2^{nd} lane) showing the consensual 3′UTR length (expected length: 1564 bps corresponding to the 1335 bps 3′UTR) and the two alternative 3′UTR lengths (341 bps and 397 bps corresponding to 3′UTR of respectively 164 and 118 bps, caused by alternative polyadenylation due to the presence of two polyA sites, wherein PCR products confirmed both by nested-PCR using spanning primer (3^{rd} lane), 1^{st} and 5^{th} lanes are DNA length ladders, 4^{th} lane is a negative control for the nested-PCR using equivalently diluted starting cDNA as template; (B) representation of the 3′ end of PCSK6 on the genome and on the mRNA transcript encompassing the exons, 3′UTRs position and lengths and the primer design used for the 3′RACE and TP-PCR and the respected splicing motif for the alternative 25^{th} exon; (C) screening of various commonly used cell lines by TP-PCR (using cDNA) showing that this alternative splicing event commonly take place in cells; (D) actinomycin-D chase performed on LNCaP cells followed by qPCR showing that transcripts having the alternative shortened 3′UTR have enhanced stability when compared to consensual ones; (E) miRNA putative sites retrieved in both 3′UTR using miRDB and RegRNA2.0 tools; (F) Luciferase reporter assay using Firefly luciferase gene carrying with the different PACE4 3′UTRs showing enhanced protein production induced by 3′UTR switching; (G) agarose gel electrophoresis of PCR product using the primer indicated on the top of each lane, the three first lane are respectively 1) the TP-PCR and each individual reaction (2 and 3), lane 4 and 5 are semi-nested PCR using a primer spanning across the junction between exon 24 and exon 25alt performed using 1/625 equivalent of the PCR products, or the cDNA equivalent (lane 6) serving as a negative control; (H) RT-qPCR analysis performed on cDNA preparation from stable PACE4-knockdown cell lines for both DU145 and LNCaP using either PACE4-FL primer (see panel G) or (I) PACE4-altCT primer (see panel G), the shRNA used to knockdown PACE4 targets exon 2 in PACE4 mRNA and for both splice variants, knockdown levels were similar.

FIG. 3 illustrates that PACE4 alternative splicing is strongly enhanced in prostate cancer specimens and correlates with tumor aggressiveness, showing in (A) TP-PCR performed on matched normal (ANCT) and cancerous prostate tissues (PCa) showing strong enrichment for the alternative exon-containing PACE4 transcripts; (B) tukey box plots of qPCR quantification of fold changes between paired tumor and ANCT samples for both consensus and alternative 25^{th} exon-carrying transcripts showing a 7.95 times stronger discrimination power for the alternative PACE4; (C) data from panel (B) presented according to tumor Gleason scores; (D) distinct intracellular distribution pattern of immunostaining in prostatic glands, PACE4-FL displaying membrane/pericellular staining whereas PACE4-altCT displays more intracellular staining, scale bars representing 200 μm.

FIG. 4 illustrates PACE4 alternative splicing and polyadenylation is dependent on CTCF-mediated exon inclusion and regulated by intra-exonic DNA methylation, showing in (A) UCSC genome browser view of PACE4 terminal exons encompassing reported ChiP-Seq enriched sequences and their associated transcription factors. miRNA from TargetScan are also shown together with the transcription levels and alignment across numerous vertebrates; (B) CpG dinucleotides methylation analyzed in paired ANCT and PCa tissues (n=13 pairs); (C) qPCR analyses of cells transfected with a siRNA targeting CTCF and a control siRNA; (D) whole cell lysate extracts of siCTCF-transfected cells; (E) PACE4 exon 25^{th} splicing index measured by qPCR in siCTCF and control siRNA transfected cells; (F) splicing indexes measured by qPCR and TP-PCR (G) in DU145 and LNCaP treated with 5-aza-dC for 72 h (equivalent of about 1.5 to 2 doubling times, respectively); (H) ChIP performed with anti-CTCF and normal IgGs in DU145 (and LNCaP) cells treated or not with 5-aza-dC for 72 h, wherein percentage of input DNA, reported as fold changes compared with the ChIP performed with normal rabbit IgG, and a positive control (Kcnq5 gene).

FIG. 5 illustrates decitabine (5-aza-dC) efficiency at hypomethylating CpG dinucleotides of interest in PCSK6 gene, showing in (A) schematic representation of the 3′ extremity of PCSK6 gene, putting emphasis on the coding and non-coding region between exon 24, exon 25 and exon 25 alt, wherein ChiP-Seq enriched regions as viewed in UCSC genome browser ChiP-Seq are represented as ovals, each CpG nucleotide analyzed is highlighted, and the inversion of 5′ and 3′ ends is shown for reading purposes; (B) DU145 and LNCaP cells (C) were treated with decitabine at the indicated concentrations for 72 h (medium and decitabine being refreshed every 24 h) and DNA was extracted and used for CpG methylation status analysis using pyrosequencing. Most CpGs were sensitive to decitabine in a dose-dependent manner, wherein DU145 showed a higher response compared to LNCaP, most likely due to their faster growth rate, decitabine being an inhibitor of DNMTs which mostly act on de novo synthesized DNA during mitosis to maintain epigenetic marks, and results are mean±SEM from at least three independent experiments.

FIG. 6 illustrates the mapping of PACE4-altCT across human tissues and different cancer types reveals a common tumor molecular switch mechanisms, showing in (A) PACE4 25^{th} exon mRNA splicing analysis across standard RNA preparation from pooled human organs either by qPCR (splicing index) or by TP-PCR; (B) splicing indexes measured across 17 types of cancerous and non-cancerous tissues cDNA; (C) quantitation of all tested PCs across the cancer types available reported as mean fold changes between normal and cancerous tissues; and in (D) IHC of PACE4 C-Termini performed on matched normal and cancerous tissues from various organs on a tissue microarray, wherein tissues sections visible are aligned for both antibodies tested, scale bars represent 100 μm.

FIG. 7 illustrates that PACE4 harboring the alternative C-terminal is equally active but differentially retained by cells, showing in (A) alternative C-Termini amino acid sequences encoded by exon 25; (B) secretion kinetic of LNCaP transfected with either V5-tagged PACE4-FL or PACE4-altCT showing distinctive levels of secretion in the medium, wherein “C” and “M” indicates Cell lysate and Medium respectively; (C) quantitative representation or the proportion of intracellular and secreted PACE4 based on fraction of total volume (for media)/protein quantity loaded on gel; (D) enzymatic activity and inhibitory profiles by the ML-peptide inhibitor of both PACE4 isoforms obtained within the conditioned media of stably expressing S2 cells, conditioned medium from non-transfected S2 cells serving as a blank; (E) Western blot of immunoprecipitation performed on the lysate used for IP-MS; (F) PACE4-derived tryptic peptides integrated area under the curve (AUC) in the 3 IP conditions showing strong enrichment in both conditions (n=6); (G) Selected proteins fold enrichment from V5 antibody immunoprecipitation (IP) performed in non-denaturating lysates of transiently expressing HEK293-FT cells compared to IP performed in non-transfected cells (n=6); (H)-(K) confocal images and quantitative co-localization analysis with V5 and RCAS1 (H), Rab5 (I), Rab7 (J) and Rab9 (K), wherein calculated values are relative to each other for each markers only (not inter-IF) considering exposition setting which varies; (M) LNCaP cell lysates from either V5-tagged PACE4-FL or PACE4-altCT following the addition of 40 μg/mL cycloheximide (CHX) showing that not only is PACE4-altCT strongly retained by cells but that its levels remains more stable over time than PACE4-FL; and (N) quantitative analysis of protein content in lysates during the CHX-chase in HEK293, DU145 and LNCaP.

FIG. 8 illustrates the PACE4 protein stability and auto-activation in transfected cells, showing in (A) after transfection with pcDNA3.1-V5 HisA encoding either PACE4-FL or PACE4-altCT, cells were treated with cycloheximide (final concentration: 40 μg/mL) and at the 0, 1, 2 and 4 h time points, PACE4 prodomain processing was determined by western blot densitometry ((B) and (C), for LNCaP and DU145 respectively) and reported as the ratio between mature/proprotein for LNCaP and DU145, the 6 h time point was omitted for this analysis as protein levels decreased considerably in some cases leading to misrepresentative quantification, and wherein the cell lysate and medium (D) and (E) were collected at each indicated time points and analyzed by western blot using both anti-V5 antibody or anti-beta actin antibody, showing representative experiment from 3 independent experiments.

FIG. 9 illustrates PACE4-altCT is responsible of sustained growth capabilities in prostate cancer cells, showing in (A) RT-qPCR analyses of PACE4 levels in stable pLenti6 transfected LNCaP cell lines; (B) Western blot of stably overexpressing LNCaP cell lines for both isoforms (without tags; using a catalytic domain oriented antibody); (C) the histogram shows the auto-activation measured by the ratio of mature divided by the sum of both pro and mature forms by densitometry analyses in stable cell lysates (n=7), blot shown is the same as in (B) but with lower exposure; (D) colony formation assays performed on the stably overexpressing LNCaP cell lines, the image showing representative stained wells; (E) RT-qPCR analyses of mRNA levels of PCs 48 h after transfection to assess knockdown levels in LNCaP cells; (F) Western blot analysis of transfected LNCaP cell lysates and serum-free conditioned media; (G) colony formation assays on transfected LNCaP plated at density of 200 cells/well for 12 days, wherein representative fields are shown above the relative quantitation relative to siNon-Target transfected cells; and (H) XTT proliferation assays performed on DU145 transfected cells 72 h after transfection.

FIG. 10 illustrates the identification and validation of GDF-15 as a PACE4-specific substrate in prostate cancer cells, showing in (A) Western blots of candidate PC substrates across the knockdown, overexpressing and inhibitor-treated DU145 and LNCaP (B) lines; (C) expression of GDF-15 in DU145 and LNCaP cells showing that mature GDF-15 is secreted in the medium; (D) GDF-15 concentrations in DU145 and LNCaP conditioned medium determined by ELISA; (E) GDF-15 cleavage analysis in the LNCaP cells panel; (F) GDF-15 concentrations in the conditioned medium determined by ELISA; (G) GDF-15 cleavage analysis in LNCaP treated with either the ML-peptide or the cell impermeable analog (PEG8-ML); (H) GDF-15 spanning peptide cleavage by PACE4 and furin monitored by HPLC, cleavage site is underlined in the spanning peptide sequence, peptide identity was confirmed by MALDI-TOF upon collection of associated fractions; (I) Western blot analysis of paired adjacent non-cancerous tissues (ANCT) and prostate tissues (PCa); and (J) a blot showing various cancer grades is shown together with the densitometric analysis of 21 tissues pairs (n=6 (3+3), 7 (3+4), 6 (4+3), 2 (5+X) X being 3 and 4).

FIG. 11 illustrates PC substrates analysis by western blots showing in (A) immunoblotting of DU145 lysates and medium in (B); and (C) LNCaP lysates and medium in (D), wherein coomassie staining of proteins from conditioned media are also presented as loading controls.

FIG. 12 illustrates the PACE4 and PACE4-altCT plasmatic concentration in PCa patients, showing in (A) ELISA-determined concentration of total PACE4 and PACE4-altCT in plasma from PCa patients and normal patients. Each individual data point is shown as a dot, and bars represent the means with SEM; (B) correlation analysis between the concentration of PACE4 and PACE4-altCT for each PCa patient, the dashed line representing the linear regression; (C) concentrations and correlation analyses of total PACE4; (D) PACE4-altCT concentrations; and (E) PACE4-altCT/total PACE4 ratios in the plasma of normal and PCa patients according to their tumor Gleason score, wherein each individual data point is shown as a dot. Data are means±SEM

### DETAILED DESCRIPTION

In accordance with the present disclosure, there is provided a tumor promoting alternative splicing isoform of PACE4 (named PACE4-altCT).

The proprotein convertases (PCs) are now recognized for their implication in malignancies through the activation of a wide spectrum of cancer-related proteins. Critical to the exploitation of PCs as drug targets is the understanding of their cellular and molecular functions. In prostate cancer, which remains the cancer with the highest incidence in men, the proprotein convertase PACE4 (PCSK6 gene name) has been proposed as an attractive target because of its documented importance in tumor progression. The PCs have been suggested as promising targets for the development of cancer therapeutics because of their positions upstream of numerous oncogenic pathways. By their endoproteolytic processing of proproteins, which includes mediators touching all key hallmarks of cancer, the activation by PCs turn out to be a limiting step between gains in term of biological activity (e.g. increased signalling by a growth factor receptor axis) following the overexpression of axis components. For this reason, if PC substrates are overexpressed by cancer cell to maximize autocrine stimulation, a concomitant increase in term of PC activity must be achieved to get full biological outcome. It is thus not surprising to see that PCs overexpression has been documented in many cancer types, however PCs reported as having increased expression are not consistent across all cancers, which may illustrate differences in tumour types and/or the absence of thorough PC scanning, as many studies often only study or assume PC activity to be assigned to a single member, namely furin.

It is provided herein that PACE4 is overexpressed in prostate cancer correlating with tumor aggressiveness, but PACE4 also undergoes a tumor promoting alternative splicing event generating a C-terminally modified isoform (named PACE4-altCT). Mapping at both mRNA and protein levels showed strong tumor reactivity in various types of cancers. Biological characterization of PACE4-altCT showed equivalent enzymatic activity to PACE4. The terminal exon replacement also favours PACE4-altCT expression and ongogenic activity in cancer cells through mRNA stabilization and auto-activation rate enhancement. PACE4-altCT initiation is sensitive to DNA methylation in the surrounding of the substituted terminal exon, as hypomethylation in this region resulted in increased splicing. PACE4-altCT is retained intracellularly and harbors a distinct localization pattern yielding increased cancer cell proliferation. PACE4-specific substrates are disclosed herein in prostate cancer, among which is growth differentiation factor 15.

Significant amount of data now indicate that the proprotein convertase PACE4 represents an attractive target in prostate cancer and in other malignancies as it is upstream of various tumor-promoting processes by the activation/maturation of various cancer-related proteins. It is provided that PACE4 overexpression in prostate cancer correlates with tumor Gleason score and that a yet unreported alternative splicing event emerge during this overexpression process and generates a C-terminally modified isoforms with pro-oncogenic features favoring PACE4 expression/activity through mRNA stabilization, enhancement auto-activation and distinct localization pattern in cells. This splice variant is susceptible to epigenetic regulation is barely found in normal tissues but common in fetal tissues and among various malignancies suggesting a tightly regulated molecular switch reinstated by cancer cells to promote PACE4 expression. Moreover, growth differentiation factor-15 is reported as the first PACE4-specific substrate in prostate cancer cells which may serve both as a PACE4 activity or as a tumor engagement biomarker.

It is provided important post-transcriptional changes that have profound effects on PACE4 mRNA and protein as well as cell trafficking and substrate processing. PACE4 alternative splicing is described as a means of regulating PACE4 expression in PCa. Analyses of PACE4 splicing by PCR and 3′RACE revealed that PCa cells utilize a splicing event leading to alternative cleavage and polyadenylation to substitute PACE4 mRNA 3′ untranslated region (3′UTR) thus promoting mRNA stability and favouring the expression of a C-terminally modified protein isoform (PACE4-altCT) which modulates its activity and retention within the cells. This yet unreported splice variant is strongly up-regulated at both mRNA and resulting protein isoform levels in PCa tissues, as well as in other cancer types such as for example but not limited to lung, thyroid and adrenal cancers. The PACE4-altCT isoform is retained intracellularly and has a different cell distribution pattern different from its parent isoform. PACE4-altCT is also accelerated in its catalytic auto-activation, further sustaining PACE4 activity increase in cancer cells as well as enhanced cell proliferative capabilities. Mapping of PACE4-alt CT across human tissues and tumours revealed a limited endogenous expression pattern, the highest levels being found in fetal tissues and various tumour types, suggesting a tightly regulated mechanism allowing sustained PACE4 activity under proliferation conditions which is taken advantage by cancer cells. PACE4 alternative splicing is further associated with distinctive intra-exonic DNA methylation between prostate normal and cancer tissues, leading to favoured terminal exon replacement following local hypomethylation in the alternative exon and DNA binding of the CCCTC-binding factor (CTCF). Finally, proteomic-based secretome analysis allowed the identification of growth differentiation factor 15 (GDF-15) as a new PACE4 substrate in PCa.

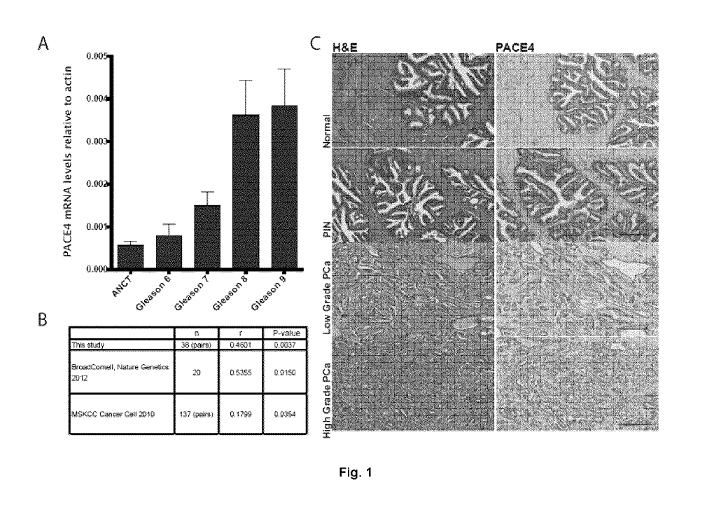

To address whether PACE4 overexpression is a stable feature across prostate tumors or an indicator of tumor aggressiveness, fresh matched normal and cancerous primary tissue specimens were obtained and used for RNA extraction and analyzes. Real-time quantitative PCR (RT-qPCR) analyzes showed that PACE4 overexpression was clearly discernable in tumor samples and that the levels tightly correlated with tumor Gleason scores (FIG. 1A, r=0.4601, P-value=0.0037). Similar analyzes using patient pre-surgery blood prostate specific antigen (PSA) levels and/or tumor staging showed no significant correlation. This observation was seconded by similar results obtained in two distinct datasets retrieved using cBioPortal for Cancer Genomics (Cerami et al., 2012, Cancer Discov, 2: 401-404; Gao et al., 2013, Sci Signal, 6, pl1) (FIG. 1B). Immunohistochemical (IHC) analyzes of specimens from different tumor grades with a polyclonal antibody targeting the protein catalytic domain corroborated these results as once again overexpression was visible with increasing levels in higher grade foci (FIG. 1C). In tumor and normal glands, PACE4 was mostly localized within prostate tissues epithelial cells and, to a lower extent, in the stromal cells. Although epithelial staining intensity readily increased along with tumoral foci grading, stromal staining remained unchanged.

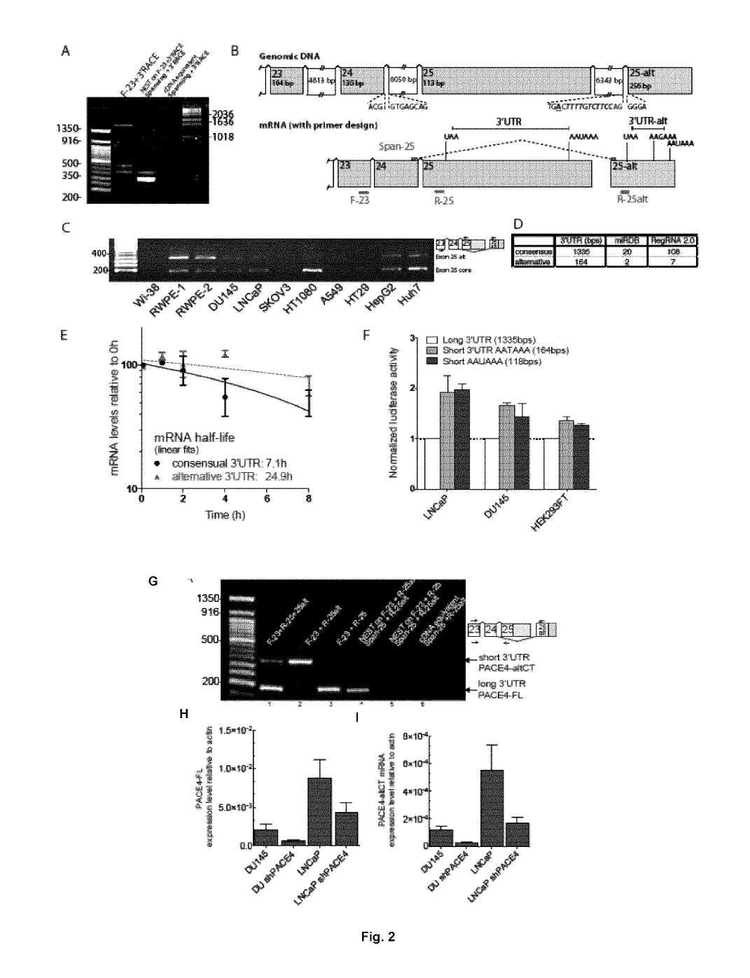

The use of alternative splicing is now recognized as a key mechanism used by cancer cells to promote the expression of genes sustaining proliferation. A more precise mechanism recently exposed is the shortening of 3′UTR regions of oncogenes and proto-oncogenes, which further allows upregulation of gene expression through the evasion from post-translational regulation mechanisms such as repression by microRNAs. PACE4 mRNA is a 969 amino acids long protein (SEQ ID NO: 1) encoded by the 186 kbs PCSK6 gene located on at the 15q26.3 locus, which is not a locus reported to be susceptible to frequent change in PCa specimens (Oncomine™ databases) (partial nucleotide sequence depicted in SEQ ID NO: 2). Analysis of PACE4 mRNA 3′ extremity by 3′ rapid amplification of cDNA end (3′ RACE) and TP-PCR in LNCaP cells revealed the presence of the consensual 1335 nt long 3′UTR (SEQ ID NO: 4) but also the presence of shorter 3′UTRs (164 nt, SEQ ID NO: 6; FIG. 2A). These product lengths, as well as nested PCRs and amplicons sequencing indicated the use of alternative polyadenylation to incorporate a distal terminal exon located 6.3 kbs downstream of the 25^{th} consensually used exon (FIG. 2B). Various cancer cell lines were evaluated for their alternative terminal exon incorporation by three prime PCR (TP-PCR; see FIGS. 2C and G), confirming this new alternative splicing event. Upon PACE4-knockdown using an exon 2-targeting shRNA, both splice variants were down-regulated confirming that both transcripts are readily derived from the same gene and harbor the same coding sections (FIGS. 2H and I). Interestingly, the cells with the highest splicing indexes were observed in the LNCaP PCa cells and the A549 lung cancer cells. Most cells expressed PACE4 transcripts containing the alternative exon (PACE4-altCT) except for the Wi38 normal lung fibroblasts, which were completely PACE4-negative.

Accordingly, compared to the native PACE4 protein which comprises exon 25 sequence consisting of:

(SEQ ID NO: 8)
ADETFCEMVKSNRLCERKLFIQFCCRTCLLAG;

PACE4-altCT essentially comprises an alternative sequence for exon 25 consisting of:

(SEQ ID NO: 9)
GDIWQRLETFWVVTTGRMYSHPVGGGQECC.

Consistent with the nature of 3′UTR and their roles in regulating mRNA stability, sequences of both consensus and alternative 3′UTR were subjected to miRNA sites prediction using RegRNA and miRDB showing striking differences in term of miRNA sites predicted in the sequences with about 90% less sites within the short 3′UTR compared to the long one. Interestingly, the miRNA regulatory sites for miR-9, -21, -124 and -543 predicted by TargetScan are all removed from PACE4 transcripts. miR-124 (a tumor-suppressor miRNA typically downregulated in PCa and miR-21 which are both validated as negative regulators of PACE4 mRNA expression thus implying miRNA evasion by this 3′UTR switch. Actinomycin-D chases followed by RT-qPCR showed that transcripts with the shorter 3′UTR were readily more stable over time when compared to consensual ones with a calculated half-life more than 3 times higher (FIG. 2E). Luciferase reporter assays performed after cloning both shorter and consensus 3′UTRs downstream of firefly luciferase in the pMIR vector were also in line mRNA stability enhancement by 3′UTR shortening as firefly production was amplified by about 2 times in transfected cell lines when the short 3′UTRs were compared to the consensus one (FIG. 2F). These data show that PACE4 mRNA can harbor two distinct 3′UTRs, consensual PACE4 transcript (PACE4-FL) or an alternative C-Terminal (PACE4-altCT).

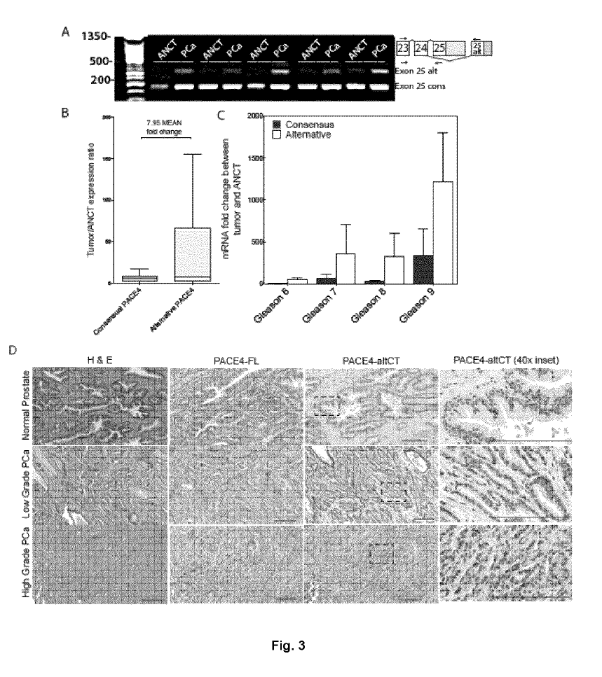

Based on the fact that PACE4 is strongly overexpressed in PCa cells with a correlation with tumor aggressiveness, paired prostate adjacent non-cancerous tissues (ANCT) and tumor tissues were analyzed for both PACE4 splice variants by TP-PCR and RT-qPCR to amplify both alternative and consensual mRNA terminating exons. PACE4-altCT mRNA was only observable in the tumor specimens with very low or undetectable levels in the ANCT matched specimens (FIG. 3A). Analyzes by RT-qPCR confirmed these results, showing that the alternative exon had about 8 times more discriminating power between tumor and normal zones with up to 250-folds difference for PACE4-altCT mRNA compared to about 30 folds for PACE4-FL (FIG. 3B). When stratified for their tumor Gleason scores (FIG. 3C), the fold changes showed increases in both spliced variants. As PACE4 has been reported to have other splice variants (Tsuji et al., 1997, Journal of Biochemistry, 122: 438-452), ANCT and cancer tissues pairs were subjected to comparative profiling of PACE4 splicing events by end-point RT-PCR to map all exon-exon junctions according to AceView database, which notably include the alternative terminal exon substitution. Among the reactions, three potential active splice sites were noted, (i) the exon 4 skipping, (ii) exon 18 skipping (also reported as PACE4-1 and PACE4-II variants, having or not the 18^{th} exon respectively (Tsuji et al., 1997, Journal of Biochemistry, 122: 438-452) and (iii) 25^{th} exon substitution. Exon 4 skipping, which would result in a truncated PACE4 catalytic domain, was previously reported as a potential biomarker for breast cancer, however in this case very little exon 4 skipping could be detected and after analysis by RT-qPCR over 25 pairs of ANCT and tumor specimens, no tumor-specificity of the weak splicing event was observed. Exon 18 skipping was found to be a very active splicing event, resulting in about 1:1 transcripts having or not the exon which encodes a short protein segment of 1.5 kDa between the RGD motif and the Cys-rich domain of PACE4. Rates of inclusion of the 18th exon were found to be constant between ANCT and tumor specimens.

Taking advantage of the two distinct C-termini encoded by the two splice variants, polyclonal antibodies were raised and affinity purified from rabbit anti-serum to discriminate both protein isoforms. Despite poor performance in western immunoblotting, the antibodies yielded interpretable in IHC as depicted by the xenograft tissues formed using PACE4-knockdown cell lines. PACE4 IHC on PCa specimens using both antibodies (FIG. 3D) showed that, as observed with mRNA, both isoforms were readily overexpressed in tumor cells and that levels increased along with tumor foci grading. On the other hand, staining for PACE4-altCT indicated a strong positive signal within the tumor epithelium with a total absence from normal epithelium. As for PACE4-FL, IHC is predominant in the prostate stroma as well as, to a lower extent, in the epithelium. Moreover, the difference of PACE4-altCT staining intensities between normal prostate glands and tumor cells was much higher than the one observed for PACE4-FL, as predicted from mRNA analysis (FIGS. 3B and C). Interestingly, the cellular distribution pattern for PACE4-altCT appeared predominantly vesicular inside the cells, which was not the case for PACE4-FL (with a more cell surface appearance).

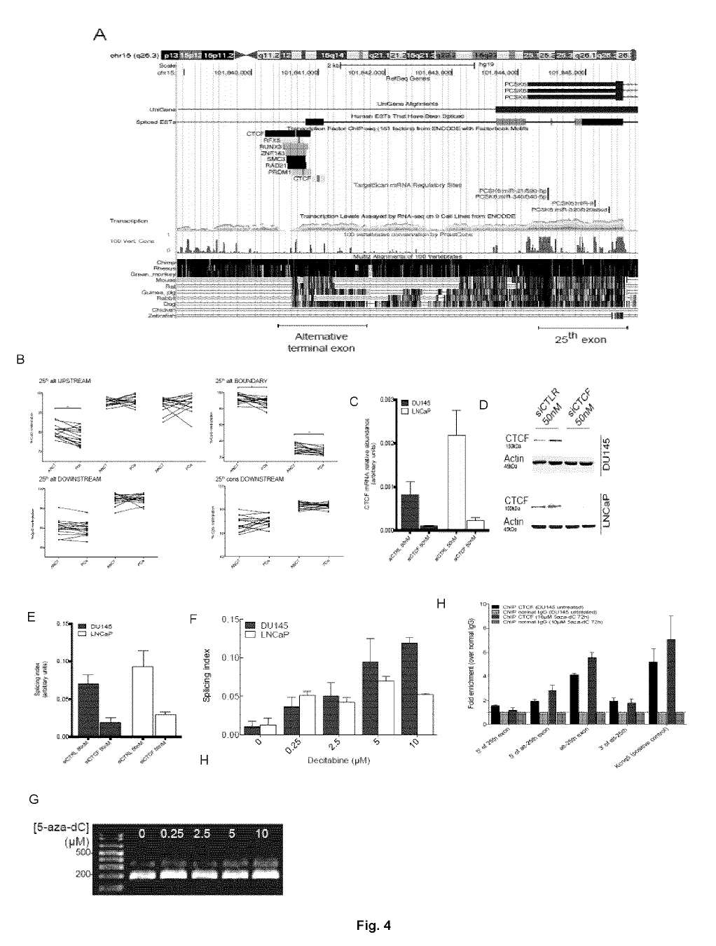

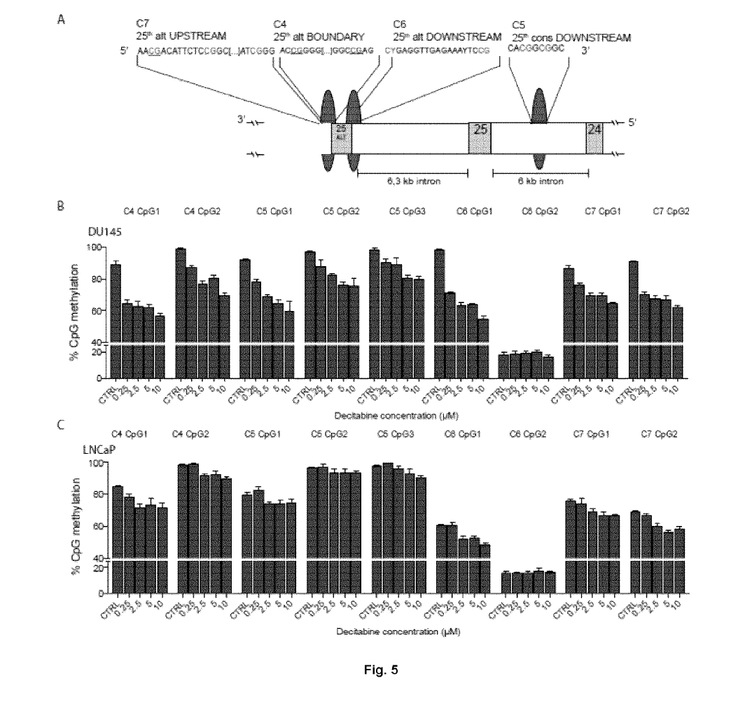

To further investigate the mechanisms regulating PACE4 terminal exon splicing, genomic sequences were visualized in UCSC genome browser to locate DNA interacting proteins in the surrounding environment of the alternative exon. Interestingly, the protein CCCTC-binding factor (CTCF) was found to have three reported binding sites according to the chromatin immunoprecipitation followed by sequencing databases (ChIP-Seq; FIG. 4A). CTCF has recently been reported to regulate upstream exon inclusion through the binding of non-methylated CpG dinucleotides in the intraexonic regions. Interestingly, 9 CpG dinucleotides were located within these CTCF ChIP-Seq enriched sequences (FIG. 5A), thus suggesting a similar regulation mechanisms. Of note, RNA-seq data assessing the transcription levels in some cell lines showed that transcription was still sustained from the consensual 25^{th} exon to the alternative 25^{th} exon (FIG. 4A). Also, the alternative 25^{th} exon is only conserved in primates (FIG. 4A) but completely absent in the other available tested vertebrates which is a common feature of alternative splicing.

DNA from both ANCT and cancerous specimens of PCa were analyzed for the methylation status of the different CpG within the alternative terminal exon and significant tumor-specific CpG hypomethylation were found in the intra-exonic and upstream CTCF binding sites (FIG. 4B). Interestingly, the observed hypomethylation was specific to these loci as the other surrounding ones were unaffected in these patients normal tissues and tumors. Such local hypomethylation indicates a tight regulation mechanisms that is mediated by locus-specific recruitment of DNA modifying or binding factors which protect DNA from methylation by DNA methyltransferases (DNMT). It has even been shown that CTCF itself could regulates DNA methylation pattern but also that cancerous or immortalized cells, when compared to normal ones, had a distinct CTCF binding landscape on the genome further encompassing the complexity of methylation-related regulation and even more when addressed in a locus-specific manner.

CTCF was transiently silenced using siRNA in DU145 and LNCaP cells and measured ration of PCAE4-altCT/PACE4-FL mRNAS determined after 72 h showed consequent reduction along with CTCF (FIGS. 4C-E), the splicing indexes reduced by 70% in both cell lines, showing the direct regulation of exon 25 substitution by CTCF. When these cells were treated with decitabine, also known as 5-aza-2′-deoxycytidine (5-aza-dC) (FIG. 5), the splicing indexes increased in a dose-dependent manner in the treated cells suggesting a relationship between the observed alternative splicing and DNA methylation (FIGS. 4F and G). ChIP experiments using an anti-CTCF antibodies demonstrated that CTCF readily binds t0 DNA within the intronic regions in 5′ and 3′ of the alternative terminal exon and within the alternative exon itself (FIG. 4H). When ChIP was carried out with decitabine-treated cells (DU145 and LNCaP), a clear enrichment for these regions could be observed in the intra-exonic and the upstream region, confirming the DNA methylation-dependent CTCF binding and exon-inclusion promotion. DU145 (FIG. 4H), showed more important differences, most likely as the results of more cell-division cycles than the LNCaP during the 72 h in presence of decitabine, yielding stronger genome hypomethylation (as seen in FIG. 5).

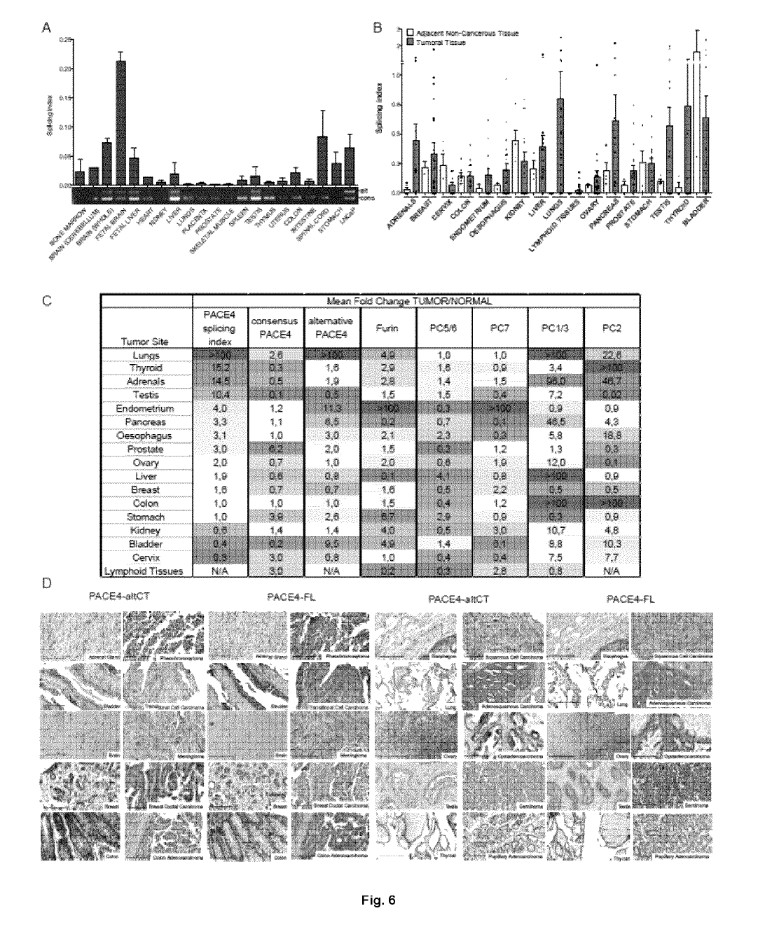

In view of the increased expression of PACE4-altCT mRNA in PCa samples, the expression pattern was observed in other tissues or other cancer types. RNA from normal human tissues were used to map both transcripts. PACE4-altCT mRNA was strongly detected in the liver, the organ reported to express the higher PACE4 levels, the testis, an organ known for its very high splicing activity and the brain/spinal cord with very little expression levels in the other organs (FIG. 6A). Interestingly, the liver, testis and brain are organs known for their higher rates of alternative splicing compared to other human tissues. Moreover, very high splicing indices were observed in fetal tissues, i.e. liver and brain, compared to the adult suggesting a tightly regulated expression mechanism. As a control, levels for every PC were also determined in these standardized RNA preparations to ensure comparability.

Splicing indexes were determined on an array of cDNA preparation from various tumor types and normal tissues, (FIG. 6B). Many cancer-types other than PCa also displayed enhanced splicing activity in the tumor specimens, notably in the lung, thyroid, adrenal and pancreatic cancers. Other PCs were also evaluated in these tumor specimen cDNA (FIG. 6C). PACE4 was found to be again the sole PC overexpressed in PCa. Moreover, PACE4, furin and PC1/3 were the only PCs displaying frequent overexpression levels across numerous cancer-types. In contrast, PC5/6 was often downregulated in tumors, which may denote an anti-tumorigenic effect of this PC in intestinal tumorigenesis. IHC analyzes using normal and tumoral specimens originating from different organs supported the mRNA mapping in normal tissues, with once again high levels of PACE4-altCT in colon and testis with the lowest observed levels in lungs, adrenals and ovary (FIG. 6D). Consistent with the observation at the mRNA level (FIG. 6B), placenta as well as lymphoid tissues tested (thymus, lymph node, lymphomas and tonsil) where all negative for PACE4-altCT by IHC whereas organs like stomach, pancreas, liver and kidney where positive to some extent. Moreover, matched tumor specimens also corroborated the expression shift observed at mRNA levels in numerous cancer-type, including lungs, esophagus, testicular and thyroid cancers to state some, thus supporting the concept that PACE4 undergoes an oncogenic alternative splicing event in PCa but also in many other types of cancer where it can act as a cancer driving factor.

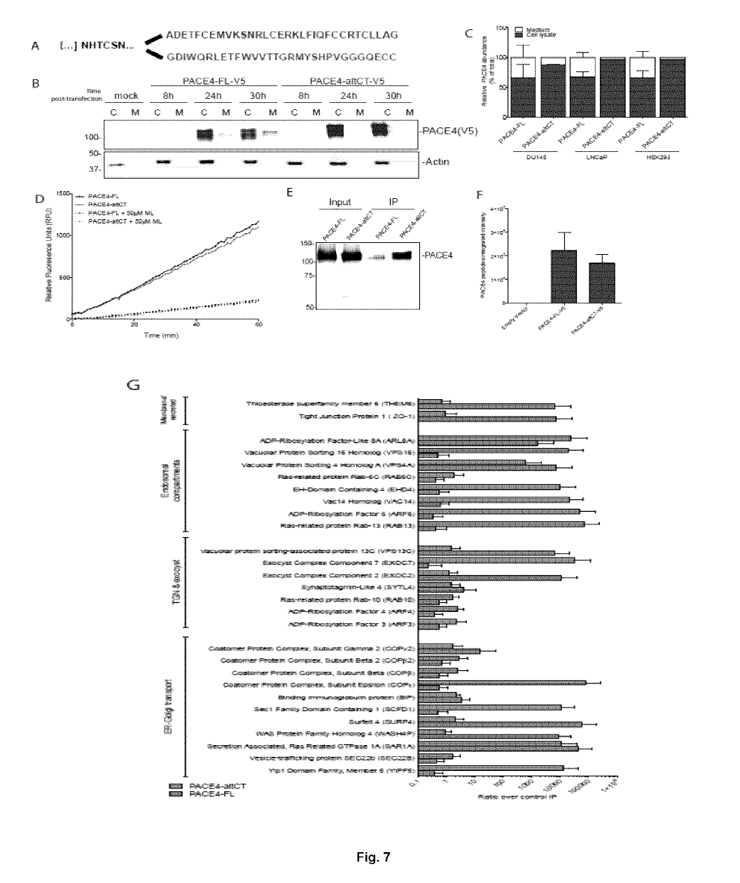

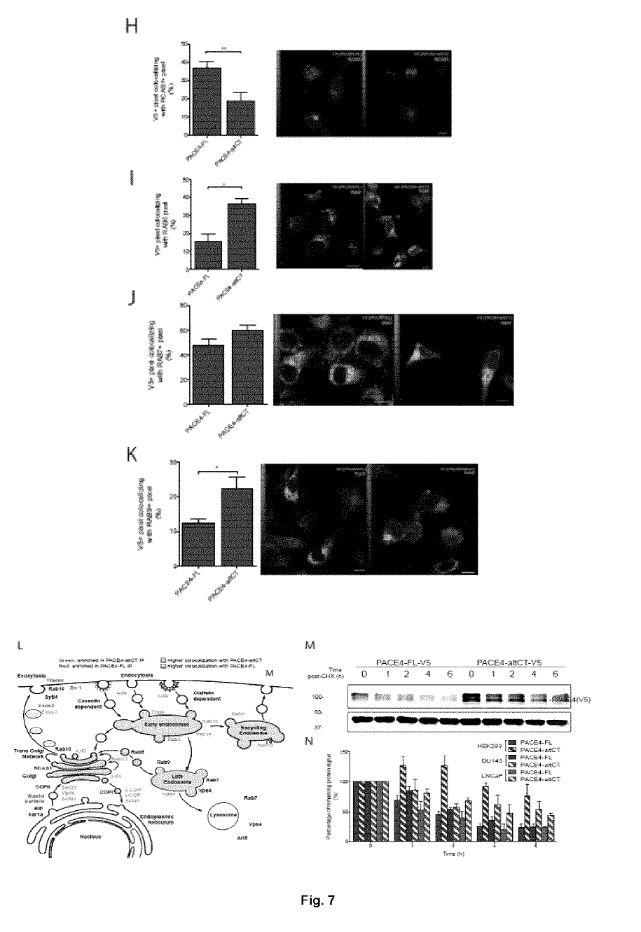

To address the question whether these two isoforms displayed equivalent functions despite their dissimilarities in their C-termini, V5 tagged-protein were expressed in cell lines (FIG. 7A). The first major distinction observed was the lack of secretion of PACE4-altCT accompanied with intracellular retention when compared to PACE4-FL which was efficiently secreted in the medium (FIGS. 7B and C). This observation correlated with the distinct cell distribution observed for each isoforms in IHC, i.e. the PACE4-FL being more accumulated at the membrane/extracellular surface compared to PACE4-altCT being restricted to the vesicular intracellular compartment (FIG. 3D). PACE4, at least PACE4-FL, is known to be secreted in the medium and to be retained in the extracellular matrix through the binding to heparan sulfate proteoglycans by its cysteine-rich domain and to be displaceable by heparin. As expected from the absence of secretion, PACE4-altCT could not be displaced by the addition of increasing doses of heparin, whereas PACE4-FL was leading to increased amount in the medium. This suggests intracellular accumulation, which was also highlighted by higher protein levels in the whole cell lysates following transfections (FIG. 7B).

Conditioned medium containing each isoforms was used to compare the enzymatic activity of both enzymes by monitoring cleavage of the fluorogenic substrate Pyr-Arg-Thr-Lys-Arg-methylcoumaryl-7-amide. C-terminal substitution did not alter activity (FIG. 7D) and both isoforms were still equally inhibited by the Multi-Leucine (ML) peptide (see WO2010/003231).

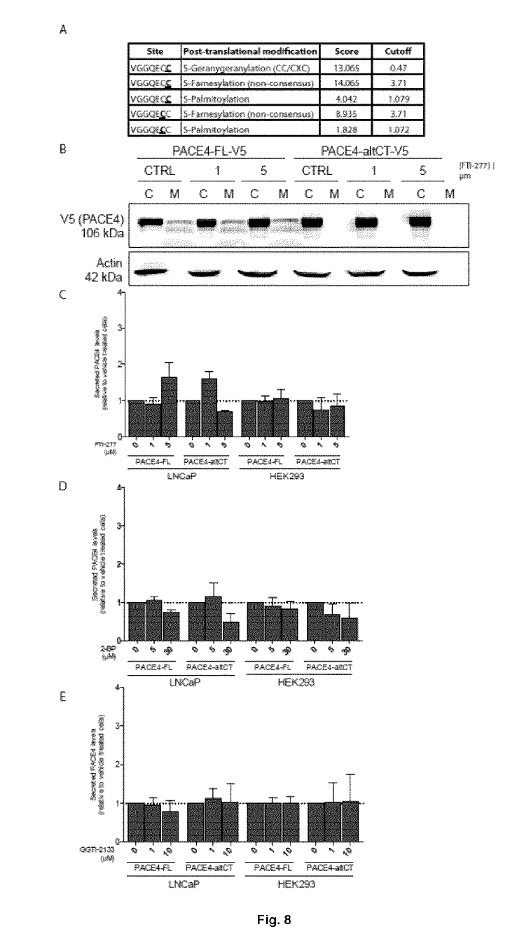

Immunoprecipitations on transiently transfected HEK293-FT cells were subjected to Sequential Window Acquisition of all Theoretical Mass Spectra (SWATH-MS)-based analysis (FIGS. 7F and G). Proteins identified in each pull-down were used to compare enrichment between the two different isoforms. Proteins typically associated with cell compartments were particularly analyzed and are shown in FIG. 7I. Both isoforms pulled-down endoplasmic reticulum (ER) proteins with similar enrichment. The two isoforms had clearly different patterns when it came to endosomal compartments-associated proteins such as Arf6, Rab13 or Vps16 with PACE4-altCT pull-down displaying the higher enriched levels for these proteins. On the other hand, PACE-FL pull-down showed a stronger association with exocyst complex-associated proteins such as Exoc2 or Vps13. Using RCAS1 (Receptor-binding cancer antigen expressed on SiSo cells) as a golgi marker, PACE4-FL was found to be almost two-times more present within the golgi compared to PCAE4-altCT (FIG. 7H). Using Rab GTPases markers, colocalization analysis showed that PACE4-altCT accumulated in Rab5-positive endosomal compartment and in Rab9-associated compartments compared to PACE4-FL (FIGS. 7I and K), suggesting a differential routing through an endosomal pathway. Considering the slight substitution caused by the splicing in the protein primary structure (FIG. 7A), PTM prediction tools were used to evaluate whether distinctive PTM motifs could be found in the alternative C-terminus compared to the FL one. The predicted PTM sites that scored the highest were S-palmitoylation, non-consensual S-farnesylation and S-geranylgeranylation on the Cys cluster added at the C-terminal extremity of the protein (SHPVGGGQECC; see FIG. 8A). Various validation assays were undertaken to verify the presence of these three PTMs; acyl-biotin exchange for S-palmitoylation, treatments of cell with, a farnesyl-transferase inhibitor (FTI-277; see FIGS. 8B and C), a palmitoyl-transferase inhibitor (2-BP; 2-bromopalmitate, see FIG. 8D) and a geranylgeranyl-transferase inhibitor (GGTI-2133; see FIG. 8E) without any significant variation in term of secretion levels.

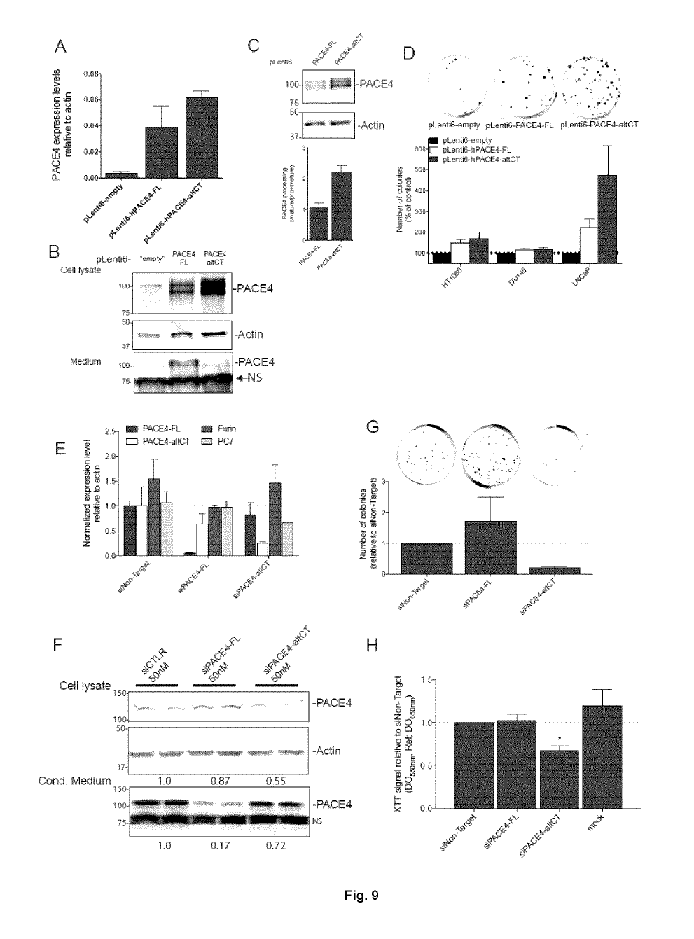

To evaluate the biological significance of these isoform-specific features, PACE4-FL and PACE4-altCT were stably expressed as untagged proteins in cell lines using lentiviral-transduction (pLenti6 vectors). Despite similar mRNA expression levels (FIG. 9A), the amounts of protein expressed and found within the whole cell lysates were much higher for PACE4-altCT compared to PACE4-FL (FIG. 9B). Very little PACE4-altCT could be observed in the medium of these overexpressing cells. Superior autocatalytic activation of PACE4-altCT was observed compared to PACE4-FL (FIG. 9C). PACE4-FL and PACE4-altCT overexpressing cells displayed enhanced growth and clonogenic capabilities as depicted by proliferation assays and colony size and quantity (FIG. 9D). PACE4-altCT overexpression yielded stronger effects, especially in LNCaP and HT1080cells, whereas the effects were modest in DU145 cells. Upon analysis of cognate PCs expression by RT-qPCR in these stable cell lines, PC7 and furin were quite affected by PACE4 overexpression suggesting cross-talks between the pathways regulating these PCs.

siRNAs were designed specifically targeting each splice variant to assess the importance of endogenous PACE4-altCT compared to its parent isoform PACE4-FL. Following transfections, each siRNA efficiently silenced its splice variant (i.e., 70-95% knockdown without affecting the other co-expressed PCs) (FIG. 9E). The siRNA targeting PACE4-altCT resulted in a stronger reduction in intracellular levels of PACE4, than the siRNA targeting PACE4-FL (i.e., 0.55 vs 0.87, respectively) (FIG. 9F). In the conditioned media, siRNA targeting PACE4-altCT had minimal effects on secreted PACE4, whereas the siRNA targeting PACE4-FL resulted in a very large decrease (i.e., >80%). These data obtained with siRNAs correlate well with previous observations concerning the differential secretion of the two isoforms.

Silencing of PACE4-altCT yielded a much stronger reduction in term of growth and clonogenic capabilities than PACE4-FL silencing, which barely affected these parameters in both LNCaP and DU145 cells (FIGS. 9G-H). These results demonstrate the role of PACE4-altCT in sustaining the growth of cancer cells.

Secreted factors have previously been suggested as the main effectors of the PACE4-related cancer cells growth phenotype upon gene silencing. For this reason, secretome analysis were performed to identify substrate candidates based on PACE4 variations. A SILAC-based proteomic approach was used to analyze the secretome content in both DU145 and LNCaP PCa cells. shNon-Target cells of both lines were cultured with heavy amino acids (¹³C₆-Arg and ¹³C₆-Lys) and compared with unlabeled (light amino acids culture medium) shPACE4 cells. Heavy amino acids incorporation in cells was confirmed using endogenously generated degradation peptides. Secretome were pooled 1:1, concentrated by acetone-methanol precipitation and fractionated by agarose-gel electrophoresis using a SageELF (Sage Science, Beverly, Mass., USA). Each fraction was analyzed by tandem LC-MS/MS. From the obtained protein identifications, secreted proteins were retrieved using ProteINSIDE and used to draw a heatmap based on light/heavy (L/H; shPACE4/Non-Target) ratio proportions for each cell line. Proteins having PC-based or PC-like processing events, determined by both Uniprot PTM/Processing data or by ProP 1.0 Server were highlighted.

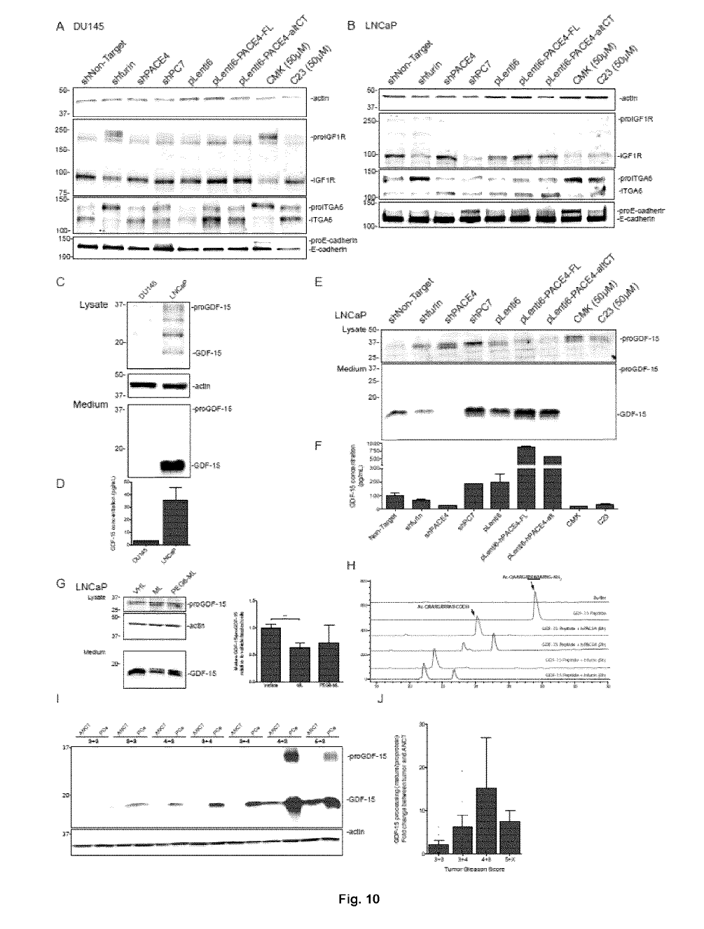

Western blotting were carried out of (i) cell lines silenced with shPACE4, shfurin and shPC7, (ii) cell lines stably expressing PACE4-FL and PACE4-altCT and (iii) cell lines treated with either the non-selective and irreversible PC inhibitor decanoyl-RVKR-chloromethylketone (CMK) or the PACE4 high affinity peptide inhibitor [dLeu]LLLRVK-amidinobenzylamide (Amba; dL-ML-Amba), herein after called C23 (Levesque et al., 2015, Oncotarget, 6: 3680-3693). In order to test the western blot arrays, known PC substrates were chosen, namely, the insulin-like growth factor 1 receptor (IGF1R) and integrin alpha-6 (ITGA6); two well-accepted furin substrates, were evaluated (FIGS. 10A and B) and E-cadherin. For both IGF1R and ITGA6, only the furin-knockdown and CMK treatments prevented the processing of their pro-forms, whereas the PACE4 knockdown and the C23 PACE4 inhibitor had no effect. In contrast, the overexpression of PACE4-FL and PACE4-altCT did increase the processing of IGF1R and ITGA6 pro-forms, highlighting the cautionary interpretation that are needed in overexpression studies. As for E-cadherin, the PC7 knockdown showed the best results to block the processing of its pro-form (FIGS. 10A and B).

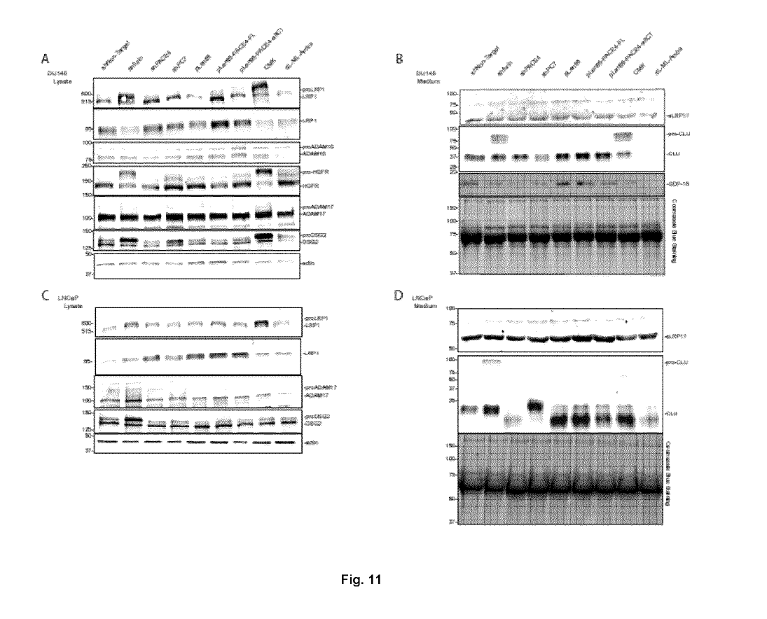

Candidate proteins detected with L/H<1 ratio in either DU145 or LNCaP that displayed a PC-based or PC-like cleavage site (FIG. 11) were further examined by immunoblotting using antibodies allowing discrimination of human pro- and mature protein forms, when available. These included low density lipoprotein receptor-related protein 1 (LRP1), hepatocyte growth factor receptor (HGFR, also known as Met), clusterin (CLU), desmoglein-2 (DSG2), ADAM10, ADAM17 and growth and differentiation factor-15 (GDF-15) (FIG. 11). By far, furin was the most important convertase for many, but not all these substrates. These included LRP1, HGFR, DSG2 and CLU. The PC7 knockdown also had some effects on these substrates, but to a much lower extent. Based on the PACE4 knockdown and the C23 inhibitor, none of these substrates are PACE4 specific. On occasion, variations in substrate levels are observed (but not conversion of pro to mature forms), explaining the differential detection in the SILAC proteomic methodology. In the case of GDF-15 (also known as prostate-differentiation factor (PDF) or macrophage inhibitory cytokine 1 (MIC-1)), a clear western blotting pattern was observed showing that this protein is uniquely processed by PACE4.

GDF-15 is known to supports both the proliferation and clonogenic potential of LNCaP cells, which is also in line with the observed phenotypes for both PACE4 knockdown and overexpressing cells. This protein is synthetized as a 35 kDa proprotein which requires a PC-based cleavage at the ARGRRRAR¹⁹⁶↓, site to generate a ˜17 kDa C-terminal mature form that associates as a disulfide-linked dimer further secreted in the medium. GDF-15 is only detected in LNCaP cells whereas DU145 express very low levels in both medium and cell lysates (FIGS. 10C and D). In the western blot array and by ELISA (FIGS. 10E and F), virtually no pro-GDF-15 could be observed in the medium shPACE4 knockdown, CMK or C23-treated cells, whereas processed GDF-15 were still highly visible after furin and PC7 knockdowns. In the PACE4-FL and PACE4-altCT overexpressing cell lines the amount of secreted GDF-15 was about 5 times higher (FIG. 10F) than pLenti6 control cells. Under all conditions, the variations of secreted GDF-15 directly correlated with the accumulation of pro-GDF-15 in the cell lysates, and not with changes in GDF-15 content, confirming that cleavage is a prerequisite for its secretion. These results demonstrated that GDF-15 is fully cleaved by PACE4 with very limited redundancy from the other co-expressed PCs and are in line with previous reports showing that in the PACE4-negative PC3 cells GDF-15 remained uncleaved. Interestingly, when placed away from the cellular environment and incubated with preparation of furin and PACE4 enzymes, the GDF-15 cleavage site isolated in a synthetic peptide (QAARGRRRARARNG) was cleaved by both PCs at the QAARGRRRAR↓, site (FIG. 10H). This results further encompass the great disparities in term of substrate cleavage redundancy among the PCs depending of the context in which it is studied.

The propeptide of GDF-15 was reported to mediates the protein retention into the extracellular matrix when it can be stored in an uncleaved form until it is cleaved. It was even observed that increased stromal stores of pro-GDF15 in clinical specimens of low-grades PCa (Gleason ≤6) were inversely correlated with tumor relapse. However, only a slight difference could be observed in the processing of GDF-15 in cells overexpressing PACE4-altCT (intracellular) and PACE4-FL (which is secreted and also located in the extracellular matrix. When treated with the cell-permeable and the PEGylated cell-impermeable version of the ML PACE4 peptide inhibitor, cleavage of GDF-15 was more susceptible to the cell-permeable version, again indicating that an important proportion of cleavage is performed inside the cells (FIG. 10G), a smaller proportion being also affected by the PEGylated peptide, but to a lower extend which may be attributable to the matrix-associated PACE4.

Interestingly, GDF-15 is highly expressed in adult prostate but it was demonstrated that mature GDF-15 is generally undetected in normal tissues in comparison with cancer zones. Analysis of pairs of non-cancerous and tumoral prostate tissues by western blot showed the same pattern (FIG. 10I) and processing quantification in tumor revealed clear trend toward increased cleavage along with tumor grading (FIG. 10J). Similar results were previously reported showing that was the presence of higher mature form by surface-enhanced light desorption and ionization (SELDI) in prostate neoplasic tissues compared to normal tissues as well as the increase of its serum concentration, which depends on the secretion of the mature form, in patients with PCa. Knowing that PACE4 expression is strongly elevated in tumor zones the relationship between these events is more than likely. Reports are even depicting that GDF-15 serum levels are strongly elevated in metastatic PCa and even make PSA sensitivity better when both proteins are measured together (Brown et al., Clin Cancer Res, 12:89-96) and a biomarker for PCa prognostic (Brown et al., Clin Cancer Res, 15: 6658-6664).

The discovery of the novel PACE4-altCT isoform, along with its strong expression in PCa specimens compared to benign prostate zones, sheds light on an important mechanism of sustained proliferation that exploits PACE4 activity to promote tumor growth. The generation of this pro-proliferative isoform with drastically different characteristics in terms of trafficking and autocatalytic activation rate appears as a sophisticated molecular switch that sustains PACE4 activity through the evasion of several regulatory elements. As with PACE4-altCT, the 3′UTR shortening of various cancer promoting genes by cancer cells had been reported across cell lines derived from numerous cancer types. Moreover, this observation is not unique to PCa cancer cells and tissues, since various cancer types also displayed strong PACE4 alternative splicing ratios suggesting an important mechanism of action. This result alone shows that PACE4-altCT (mRNA or protein) is a biomarker for PACE4-dependent cancers.

A comprehensive analysis comparing the expression levels of all PCs among such a broad array of cancer types (FIGS. 6B and C) demonstrates that some PCs, such as PC5/6, are in most cases down-regulated or unchanged whereas others such as furin and PACE4 being generally overexpressed but not in always in a concomitant manner. The mapping performed on normal human tissues (FIGS. 6A and D) is revealing, especially when comparing the levels of each PC measured alongside by RT-qPCR. The most impressive results from this mapping being the very limited expression pattern of PACE4-altCT, with considerable levels in testis and liver, but more importantly in fetal tissues (brain and liver; FIG. 6A) thus suggesting a mechanism used in development and further reinstated by cancer cells to sustain their growth. This fact is further reinforced by the regulation of this mechanism by intragenic epigenetic modifications and the further regulation of the binding of CTCF, and possibly other DNA-binding factors reported to bind this chromatin segment. It is however interesting to see that only human and primates have this alternative exon conserved in the genomic point of view whereas all other species lack a similar genomic environment allowing terminal exon substitution. This is coherent with the observations showing that alternative splicing frequencies decline rapidly when the evolutionary distance from primates increases, thus suggesting that studies performed in murine models may lack a significant element concerning PACE4 biology. Indeed, the discovery of this novel spice variant of PACE4 revise considerably the understanding of the cumulated literature since in all cases PACE4-FL was used to get to conclusions and it is now clear that most conclusions cannot be directly applied because of the intracellular localization disparities. Moreover, the absence of this isoform in murine and rodent models also bias some data interpretation across species. The discovery of this intracellular isoform also refines the working model since it was previously demonstrated using polyethylene glycol-modified peptide that PACE4 inhibitors antiproliferative activity over PCa cells was strongly dependent on their cell penetration properties. Knowing this and combined with the observation that PACE4-altCT exhibit much higher growth stimulation capabilities it is disclosed that the effecting target of the ML peptide is PACE4-altCT, even if both isoforms are equally inhibited by the inhibitor in vitro. This is further highlighted by i) the strong intracellular retention of the ML peptide into cancer cells with respect to their PACE4 levels and ii) strong xenograft uptake of the ML peptide when administered to tumor-bearing mice which would be hard to conciliate if the target was purely a secreted protein like PACE4-FL is. However, this does not implies that all PACE4 substrates are cleaved inside the cells, as encompassed by the case of the B isoform of the insulin receptor which is cleaved by PACE4 at the cell surface in furin-deficient conditions, findings which were also determined using inhibitors with distinct cell penetration properties.

PACE4 prodomain removal being the prerequisite step prior to the sequential export from the ER and further transport to the golgi and the TGN (and ultimately the extracellular space) where calcium and pH conditions permits higher activity, the accumulation of PACE4-altCT into the secretory pathway is coherent with the observations that PACE4-altCT displays strong enrichment in term of intracellular mature form (FIG. 9C) and comparable activity in overexpressing cells when compared to PACE4-FL (FIGS. 10A, B, E and F). This different routing may be due to the Cys-rich domain re-arrangement (which usually contains 44 Cys residues) following the substitution of 5 Cys for 2 Cys in the alt-Cterminal, which may influence protein interactions in the secretory pathway in the ER, the golgi or the TGN.

PACE4 seems to be the sole PC overexpressed in PCa (FIG. 6C) and cancer cells rely on this single PC for sustaining their proliferation. As disclosed herein, a silencing approach was used to eliminate the possible drawbacks associated to overexpression to identify GDF-15 as a direct PACE4 substrate in PCa cells. Its activities on cell are vast; in breast cancer cells GDF-15 expression following exposure to radiation is known to protect them from radiation-induced cell death and in malignant melanomas, GDF-15 overexpression leads to sustained neovascularization. Interestingly, impaired vasculature development is also an observed phenotype of PACE4-knockdowned or PACE4-inhibitor treated xenografts. Despite not being substrates of PACE4, many of the secreted proteins were detected which were detected with drastic levels reduction in the conditioned medium of the shPACE4 cells are of great interest. Proteases such as cathepsins D, Z, B and H, which act as key regulators of tumor angiogenesis and extracellular matrix remodeling, as well as angiogenic factors (e.g. ephrin-A1 and angiogenin) are part of these interesting downstream molecules which are most likely the results of the attenuated processing of substrates upstream of signaling pathways.

Having such PACE4 activity biomarkers measurable in the serum allows to assess target engagement in pharmacological intervention using PACE4 inhibitors but also as an indirect way to measure PACE4 levels in the organs through a simple blood sample. GDF-15 is an ideal marker for such analysis since its expression is strongly limited to the prostate (and to the placenta). On the diagnostic/prognostic point of view, PACE4, or more precisely PACE4-altCT represent a marker directly, either by IHC or directly as serum marker.

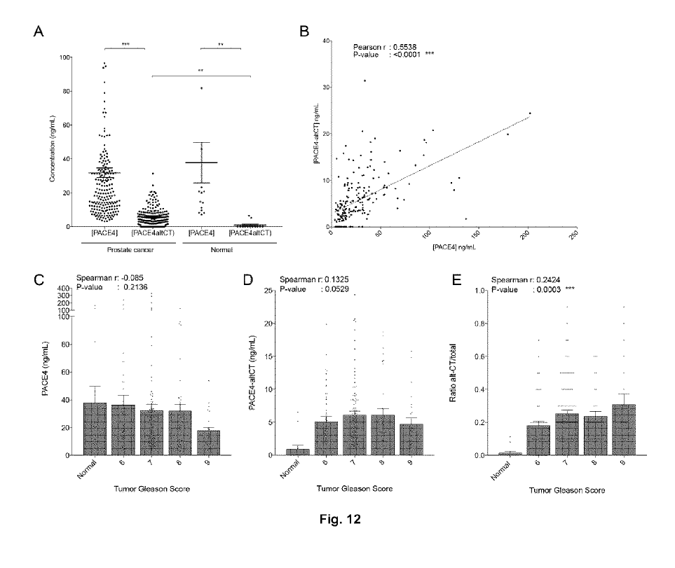

Furthermore, levels of PACE4-altCT in plasma correlate with tumor Gleason score. When plasma level of PACE4-altCT collected from patients just prior to radical prostatectomy showed as expected total PACE4 levels were much higher than PACE4-altCT levels, with averages of 31 ng/mL and 5.4 ng/mL, respectively (FIG. 12A). Total PACE4 levels in plasma from normal and PCa patients did not differ (averages of 31 vs 37 ng/mL, respectively), whereas PACE4-altCT levels were much more elevated in PCa compared to normal patients (averages of 5.4 vs 0.9 ng/mL, respectively). Correlation analysis indicated that the levels of both isoforms were correlated with each other (Pearson r: 0.5538, P-value: <0.0001; FIG. 12B) but not with PSA levels. Total PACE4 plasmatic concentrations did not correlate with tumor Gleason scores (FIG. 12C). However, PACE4-altCT levels displayed a clear tendency to correlate with tumor aggressiveness (Spearman r: 0.1325, P-value: 0.0529; FIG. 5D). Upon normalizing the portion of PACE4-altCT over the total circulating PACE4 (as a ratio PACE4-altCT/PACE4), a clear and significant correlation with tumor Gleason score was determined (Spearman r: 0.2424, P-value: 0.0003; FIG. 5E).

The development and validation of PACE4-altCT specific ELISA permitted the confirmation that PACE4-altCT is not only increased in PCa tissues but also that it can be found in the bloodstream.

It is described herein a method for detecting prostate cancer in a subject comprising the steps of obtaining a biological sample from the subject; and detecting said prostate cancer by detecting the presence of PACE4-altCT in said biological sample.

The method described herein can comprise the further steps of contacting an analyte specific reagent specifically binding to the PACE4-altCT with the biological sample under conditions so as to allow the formation of an analyte-PACE4-altCT complex, and detecting prostate cancer by detecting the analyte-PACE4-altCT complex.

The term “analyte specific reagent” or “ASR” refers to any molecule including any chemical, nucleic acid sequence, polypeptide (e.g. receptor protein) or composite molecule and/or any composition that permits quantitative assessment of the analyte level. Accordingly, the analyte can be an antibody, a peptide, a primer or a probe.

The term “specifically binds” as used herein refers to a binding reaction that is determinative of the presence of PACE4-altCT.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

To produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with cancer cells and the monoclonal antibodies can be isolated.

Specific antibodies, or antibody fragments, reactive against particular target polypeptide gene product antigens, can also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

It is thus encompassed an analyte, such as for example a monoclonal or polyclonal antibody, specifically recognizing PACE4-altCT. Accordingly, in an embodiment, the probe recognizes the alternative exon 25 present in PACE4-altCT depicted in SEQ ID NO: 8.

More particularly, said antibody is a monoclonal or a polyclonal antibody. In another embodiment, said antibody is a mouse antibody, a goat antibody, a human antibody or a rabbit antibody. Also encompassed is a humanized antibody specifically recognizing PACE4-altCT. The antibody described herein can comprises an epitope binding fragment selected from the group consisting of: Fv, F(ab′), or F(ab′)2.

More particularly, the antibody described herein specifically binds to an epitope comprising the amino acid sequence set forth in any one of SEQ ID NOs: 18, 23, 24, 25, and 26.

The term “probe” as used herein refers to a nucleic acid sequence that comprises a sequence of nucleotides that will hybridize specifically to a target nucleic acid sequence encoding PACE4-altCT. For example the probe comprises at least 10 or more bases or nucleotides that are complementary and hybridize contiguous bases and/or nucleotides in the target nucleic acid sequence. The length of probe depends on the hybridization conditions and the sequences of the probe and nucleic acid target sequence and can for example be 10-20, 21-70, 71-100, 101-500 or more bases or nucleotides in length. The probes can optionally be fixed to a solid support such as an array chip or a microarray chip.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

Thus, the probe specifically recognizing PACE4-altCT can be a primer, an oligonucleotide, a siRNA molecule for example which specifically recognises PACE4-altCT. More particularly, the probe encompassed herein specifically binds to PACE4-altCT. For example, the probe described herein can specifically bind to a nucleotide sequence comprising SEQ ID NOs: 5 or 6. In another embodiment, the siRNA molecule encompassed herein comprises the nucleotide sequence set forth in SEQ ID NOs: 15 or 16.

The method described herein can further comprise the step of applying a detection agent that detects the analyte-PACE4-altCT complex.

A “detectable label” or “detectable agent” as used herein means an agent or composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The detection agent can be detected by techniques known in the art such as Western blot, ELISA, immunoprecipitation followed by SDS-PAGE, immunocytochemistry, immunohistochemistry, PCR, or RT-PCR. Thus, the PACE4 splicing isoform detected can thus be a protein or a nucleic acid molecule.

Accordingly, using antibodies raised against any of the discussed epitopes, Western blots could be carried out on blood-derived specimens (crude or concentrated) to detect PACE4-altCT proteins.

Furthermore, using antibodies raised against any of the discussed epitopes, ELISA could be carried out on blood-derived specimens (crude or concentrated) to quantify PACE4-altCT proteins.

In addition, mass spectrometry (MS) based quantification using multiple reaction monitoring (MRM) resolves many of the reported issues (Makawita and Diamandis, 2010, Clin Chem, 56: 212-222) as it allows high structural specificity and high multiplexing capacity (Anderson and Hunter, 2010, Mol Cell Proteomics, 5: 573-588). MRM quantification is performed by the combination of liquid chromatography (LC) and highly sensitive triple quadrupole MS. To date, the most important limitation of MS technology has been sensitivity, only reaching the mg/L quantification in blood mostly because of interference from abundant proteins. Using proper sample preparation; (i) sample fractionation (Fortin 2009, Anal Chem, 81: 9343-9352), (ii) depletion of abundant proteins (Anderson and Hunter, 2010, Mol Cell Proteomics, 5: 573-588) and (iii) affinity capture of target protein (Nicol et al., 2008, Mol Cell Proteomics, 7: 1974-1982) or target peptide (named: Stable Isotope Standards and Capture by Anti-Peptide Antibodies; SISCAPA) (Anderson et al., 2004, J Proteome Res, 3: 235-244), the limit of quantification (LOQ) for protein (e.g. PSA (Keshishian et al., 2007, Mol Cell Proteomics, 6: 2212-2229) has now improved 1000 fold (mg/L to μg/L) with percent coefficient of variation (% CV) down to 2.8% with results comparable to ELISA assays (Fortin 2009, Anal Chem, 81: 9343-9352). In one report, serum PACE4 detection, was obtained using the SISCAPA method (Klee et al., Clin Chem, 2012, 58: 599-609).

Detection of PACE4-altCT could be performed by affinity enrichment of PACE4 using an antibody that as affinity to all PACE4 splicing isoforms or to PACE4-altCT, using anti-PACE4 peptide (SISCAPA) for selective detection of PACE4 splicing isoforms. For all those methods PACE4 splicing isoforms would be quantified and detected by selective enzymatic digestion and LC-MS/MS analysis.

Alternatively, antibody free methodology could by applied using selective enrichment of target peptide. After PACE4 digestion by selective enzymes (eg. Trypsin, chymotrypsin), the peptide of interest could be selectively enriched by reproducible orthogonal liquid chromatography or by ion exchange or polymeric ion exchange solid phase extraction followed by LC-MS/MS quantification.

In still a further aspect, the disclosure provides a method of selecting prostate cancer subjects for a clinical trial. The method comprises determining a subject's test PACE4-altCT expression profile and prognosis according to a method as described herein; and including or excluding the subject in the clinical trial based on their prognosis.

In a further aspect, it is provided a method for prognosis of a subject having received an initial diagnosis of prostate cancer.

As used herein “prognosis” refers to an indication of the likelihood of a particular clinical outcome, for example, an indication of likelihood of recurrence, metastasis, and/or death due to disease, overall survival or the likelihood of recovery and includes a “good prognosis” and a “poor prognosis”.

As used herein, “good prognosis” indicates that the subject is expected e.g. predicted to survive and/or have no, or is at low risk of having, recurrence or distant metastases within a set time period, for example five years after initial diagnosis of prostate cancer.

As used herein, “poor prognosis” indicates that the subject is expected e.g. predicted to not survive and/or to have, or is at high risk of having, recurrence or distant metastases within a set time period, for example five years of initial diagnosis of prostate cancer.

As used herein, the term “recurrence” refers to the reappearance of cancer, such as prostate cancer within a set period of time from initial diagnosis, for example 5 years.

As used herein, the term “disease free survival” refers to no reappearance of cancer, such as prostate cancer within a set period of time from initial diagnosis, for example 5 years.

A further aspect of the disclosure includes a method of identifying agents for use in the treatment of prostate cancer. Clinical trials seek to test the efficacy of new therapeutics. The efficacy is often only determinable after many months of treatment. The methods disclosed herein are useful for monitoring the expression of PACE4-altCT associated with prognosis. Accordingly, changes in PACE4-altCT expression levels which are associated with a better prognosis are indicative the agent is a candidate as a chemotherapeutic.

Accordingly in an embodiment, the disclosure provides a method for identifying candidate agents for use in treatment of prostate cancer.

As used herein “sample” refers to any subject's sample, including but not limited to a fluid, cell or tissue sample that comprises tumor associated stromal cells, which can be assayed for gene expression levels, particularly genes differentially expressed in patients having or not having prostate cancer. The sample includes for example bulk tumor, isolated stromal cells, a biopsy, a resected tumor sample, a frozen tissue sample, a fresh tissue specimen, a cell sample, and/or a paraffin embedded section or material.

The term “subject” also referred to as “patient” as used herein refers to any member of the animal kingdom, preferably a human being.

### Example I

### Cell Culture and Proliferation Assays

Cell lines were obtained and cultured in the following conditions: DU145 (American Type Culture Collection; ATCC, Mannasas USA, RPMI 1640; 5% fetal bovine serum; FBS, Wisent Bioproducts, St Bruno, QC), LNCaP, PC3 and HT-29 (ATCC, RPMI 1640; 10% FBS), SKOV3 (ATCC, DMEM-F12K; 10% FBS), HEK293-FT (Life Technologies Inc., DMEM; 10% FBS, 500 μg/ml Geneticin), Huh7 and A549 (ATCC, DMEM; 10% FBS), HT1080 and HepG2 (ATCC, EMEM; 10% FBS). Stable knockdown cell lines were the same as reported in (Couture et al., 2012). S2 cells were cultured and used for production of recombinant PC as described in Fugere et al. (2002, Journal of Biological Chemistry, 277: 7648-7656).

For actinomycin-D (Sigma Aldrich) or cycloheximide (Sigma Aldrich) treatments, compounds were first dissolved in DMSO, and diluted to a final concentration of 5 μg/mL and 40 μg/mL in the culture medium respectively. Actinomycin-D treatment never exceeded 8 h, which is the time-frame prior to early apoptosis induction. For treatments wit 5-aza-2′-deoxy-cytidine (Sigma Aldrich), compound was first dissolved in DMSO and further diluted prior to addition to cell culture medium, which was changed every day and replaced with fresh one containing the compound for a total exposure of 72 h. For DNA transfections, cells were lipofected using Lipofectamine 3000 (Invitrogen) and DNA plasmids (purified using QIAgen plasmid purification kit following manufacturer guidelines), if not stated otherwise cell were lysed in lysis buffer (Tris-HCl 50 mM, NaCl 150 mM, SDS 0.1%, Na-Deoxycholate 0.5%, Triton-X100 1% and NP-40 1%) containing 1× protease inhibitor (Roche Diagnostics). siRNA (Cell Signaling Technologies) were transfected using Lipofectamine RNAiMax (Life Technologies) following manufacturer guidelines. siRNA were purchased from Cell Signaling Technologies (CTCF siRNA I #6265 and Control siRNA #6568). Cell were lysed 48 h post-transfection either for RNA or protein extraction.

For PACE4 secretion assays, cells were plated at equal densities in 6-well plates for 24 h in complete medium. 24 h later, the medium was replaced by the minimal volume required of fresh culture medium (600 μL without FBS) and the cells were either allowed to secrete for the indicated time (for secretion kinetics) or (for inhibitor treatments) allowed to secrete for an additional 24 h in the presence of the indicated concentrations of agents (heparin (Sandoz, Niirnberg, Germany), 2-bromopalmitate (Sigma Aldrich), FTI-277 (Sigma Aldrich), GGTI-2133 (Santa Cruz Biotechnology Inc., Santa Cruz, Calif., USA)

cDNA were cloned into either pAc5.1-V5-HisA, pcDNA3.1-V5-HisA or pLenti6 vectors encoding PACE4 splice variants which were obtained through gene synthesis (GeneArt, Thermo Fisher Scientific). For transient expression, cells were transfected using pcDNA3.1-V5-HisA constructs (2.5 μg DNA in 6 well-plate) using Lipofectamine3000 reagent (Thermo Fisher Scientific). For stable overexpressing mammalian cell lines, cell were transduced with lentiviral preparation produced as described in (D'Anjou et al., 2013) and further selected using blasticidin (HT1080 and DU145: 5 μg/mL, LNCaP: 20 μg/mL).

For substrates analysis, cell lysates were prepared by plating equal number of cells in p100 mm plates in complete medium. 24 h later, medium was replaced by 6 mL of serum-free fresh medium (with treatment if indicated; 50 μM of dL-ML-Amba or dec-RVKR-CMK (Bachem, Torrance, Calif.) and cells were further incubated 48 h. Medium was then collected and centrifuged at 1,000×g for 10 min at room temperature to remove any floating cells, aliquot of medium were then taken (800 μL), flash-frozen in liquid nitrogen, lyophilized overnight, restituted in 100 μL of Laemmli buffer: 8M urea (1:1) and boiled for 5 min until complete resuspension. 25 μL of concentrated medium were loaded on SDS-PAGE (equivalent of 200 μL of culture medium). Cells were carefully washed with PBS and lysed from cell pellet (resulting from 1,000×g centrifugation) using radio-immunoprecipitation assay buffer (RIPA) as described in (Couture et al., 2012). Samples were incubated 20 min on ice and further centrifuged 30 min at 13.000 rpm at 4° C. Protein concentration was determined by bicinchoninic acid assay (Pierce) to load 15 μg on polyacrylamide gels. 1-actin was used as a loading control, for conditioned media, a Coomassie blue staining was routinely performed to control for protein loading.

For proliferation assays, cell were plated in 96 wells-plates at identical densities and after 72 h, metabolic activity was measured using 3-(4,5-dimethylthiazol-2-yl)-2,5-diphenyltetrazolium bromide (MTT) reagent as described in Levesque et al. (2012, Journal of Medicinal Chemistry, 55: 10501-10511). For colony formation assay, cell were plated at low densities (50 cells for HT1080, 100 cells for DU145 and 500 cells for LNCaP) and allowed 10 days to form colonies in complete medium before being stained with crystal violet and manually counted. Stained plates were also scanned (Odyssey Imager, LI-COR Biosciences) and colony area were determined using ImageJ software.

For activity assays, medium collected from stable S2 cells culture expressing the PACE4 isoforms were collected and buffer-exchanged on Amicon-Ultra 30K spinnable units (Millipore) with PACE4 activity buffer (bis-Tris 20 mM pH 6.5, 1 mM CaCl₂). Western blotting confirmed equivalent PACE4 amount within each preparation which were further used for activity assays performed as described in Levesque et al. (2012, Journal of Medicinal Chemistry, 55: 10501-10511). For ML-peptide inhibition, 50 μM of inhibitor were added to the activity assays. Identical preparation from wild type S2 cells were used as blank.

For confocal microscopy, HT1080 cells were plated on poly-L-lysine coated glass coverslip and further transfected with pcDNA3.1-V5-HisA vectors. 48 h after transfection, cells were fixed in 4% paraformaldehyde in PBS for 15 min, permeabilized with blocking buffer (PBS; 0.3% Triton-X-100; 2.5% goat serum; 1% BSA) for 1 h at room temperature. Cells were then incubated overnight with the primary antibodies at 4° C. Fluorescent secondary antibodies (AlexaFluor-488 and -594 antibodies, ThermoFisher) were further used (1 h incubation, room temperature) followed by DAPI (300 nM; 10 min, room temperature) and final mounting with SlowFade (Invitrogen). Cells were examined with an Plan Apo 60× oil immersion objective NA 1.42 on inverted spectral scanning confocal microscope FV1000 (Olympus, Tokyo, Japan). In order to avoid the cross-talk between the emitted Alexa Fluor 488 and Alexa Fluor 594 fluorescence was collected sequentially. Images were acquired during the same day, typically from 7-15 cells of similar size from each experimental condition using identical settings of the instrument. For the quantitative analyze of the overlap quadrant ranks (thresholds) were placed forming background (C), red-only (D), green-only (A) and colocalization areas (B). Colocalization index were calculated as (B)/(B+D), and % of colocalization as (B)/(B+D)×100.Quantitative analysis was performed on minimally 7 size-matched cells for each experimental condition.

### Example II

### Fresh Tissues Dissection and Assays

Prostate tissues used for RNA extraction were freshly (typically within 30 min) dissected from prostate specimen obtained from radical prostatectomies performed at the Centre Hospitalier Universitaire de Sherbrooke. Patients agreed to participate and freely signed a consent form and the research protocol was approved by the Institutional Review Committee for the Use of Human Resected Material at the Centre Hospitalier Universitaire de Sherbrooke. Tissues were frozen at −20° C. with OCT compound (Tissue-Tek; Miles Scientific) and slices of 5 μm were cut and immediately fixed in formalin to perform hematoxylin-eosin staining for pathological examination. Tumor zones were delimitated together with the adjacent non-cancerous tissues by a clinical pathologist and dissection was performed accordingly. Dissected tissues were washed with nano-pure RNase free water (Wisent) to remove all apparent traces of OCT compound. Tissues were then powder-crushed in liquid nitrogen and RNA extraction was performed using QIAgen RNeasy spin columns (QIAgen, Valentia, Calif., USA) following manufacturer instructions. RNA integrity was assessed by analysis using Agilent Bioanalyser with RNA Nano Chips (Agilent Technologies, Palo Alto, Calif., USA).

1 μg RNA was DNase I-treated (Invitrogen), reverse-transcribed using Superscript II reverse transcriptase (Invitrogen), and RNase H-treated (Ambion, Austin, Tex.) before quantitative PCR performed using a Stratagene Mx3005P instrument. Relative expression levels were calculated using β-actin as a reference gene with the formula (1+amplification efficiency)^{-(CT)}. Experiments were done at least in three independent experiments (n=3).

PCR experiments flanking all possible exon-exon junctions were designed. In addition, alternative splicing events were covered by at least two independent reactions, where possible, based on the AceView database containing most EST transcripts. The AceView transcript sets were mapped into the LISA database and the LISA automatically generated a splicing map. When possible, the design was such that predicted amplicon sizes fell within the 100 to 400 bp range. The lower limit of 100 bp was set to avoid an overlap with primer and primer-dimer signals. As described in Klinck et al. (2008, Cancer Research, 68: 657-663), end-point PCR reactions were done on 20 ng cDNA in 10 μL final volume containing 0.2 mmol/L each dNTP, 1.5 mmol/L MgCl₂, 0.6 μmol/L each primer, and 0.2 units of Taq DNA polymerase. An initial incubation of 2 min at 95° C. was followed by 35 cycles at 94° C. 30 s, 55° C. 30 s, and 72° C. 60 s. The amplification was completed by a 2-min incubation at 72° C. PCR reactions are carried out using a liquid handling system linked to thermocyclers, and the amplified products were analyzed by automated chip-based microcapillary electrophoresis on Caliper LC-90 instruments (Caliper LifeSciences). Amplicon sizing and relative quantitation was performed by the manufacturer's software, before being uploaded to the LISA database.

Rapid amplification of cDNA 3′ends was done using LNCaP total RNA which was reverse transcripted with 10 μM of cDNA cloning primer (GGCCACGCGTCGACTAGTACTTTTTTTTTTTTTTTTTV, SEQ ID NO: 40; IDT, Coralville, Iowa). 3′UTRs were further amplified by PCR (35 cycles; 95° C. 30 sec, 62° C. 30 sec, 72° C. 3 min) using 1 μL of cDNA with F24 (0.2 μM) as a gene specific primer (ACCCAGAAGAGATGCCGG, SEQ ID NO: 41) and 3′RACE primer (0.05 μM; GGCCACGCGTCGACTAGTAC, SEQ ID NO: 42). For nested-PCR, PCR product was diluted 1:1000 and 1 μL was used in side by side with an equivalent dilution of the original cDNA to serve as a control. DNA bands were electro-eluted out from ethidium bromide stained agarose gels, and used for sequencing after ethanol precipitation. 3′UTRs were cloned by nest-PCR using Q5 high-fidelity polymerase PCR products (New Englands Biolabs, Canada) with primers containing AscI (starting just after stop codon) and XbaI (finishing just after poly-adenylation signal) respectively. 3′UTR were inserted into pMIR reporter vector (OriGene Technologies, Inc.; Rockville, Md.) and after used for transfection in cells after vector sequencing using 4 ng DNA per well in 24-well plates. Luciferase activities were measured 24 h later according to the manufacturer's instructions by first removing the medium and then adding Dual-Glo assay solutions (Promega) using a SIRIUS luminometer (Berthold Detection Systems, Pforzhein, Germany). Luminescence was normalized to protein content in samples as determined by bicinchoninic acid assay (Pierce). For miRNA alignment, sequence of the 3′UTR were submitted RegRNA and miRDB.

### Example III

### Antibodies Used and Generation and Immunohistochemistry

Rabbit polyclonal antibodies were raised and purified from serum on peptide coated chromatographic column (Pacific Immunology, Ramona, Calif.). For immunohistochemistry, slides with 4 μm tissue slices were incubated 5 min in each of the following solutions at room temperature: 2× xylene, 2× ethanol 100%, 95%, 85%, 70%, 50%, 30%, 2× UltraPure Water, 10 mM citrate buffer pH 6 and further autoclaved in 10 mM Citrate buffer pH6 for 45 min (16 psi, 250° F.). After cool-down at room temperature, IHC was performed using the Peroxidase Detection Kit (Pierce). Tissues sections were incubated overnight at 4° C. with the primary antibodies diluted in BSA 5% in TBST and further incubated following washes with a secondary HRP-conjugated antibody (Anti-Rabbit HRP from BioRad; diluted accordingly in TBST 5% BSA, 1/500). Slides were then counterstained in Harris hematoxylin (Sigma-Aldrich). For blocking peptide co-incubation, 30 μg of epitopic peptide were added to the primary blotting solution. The xenografted line tissues used for antibody validation are those described in Longuespee et al. (2014, Translational Oncology, 7: 410-419). For immunohistochemistry in other cancer types, Paraffin Tissue Array was obtained from Biochain (Newark, Calif.) on which primary tumors as well as matched non-tumoral tissues were present.

Protein were submitted to electrophoresis and further transferred to a nitrocellulose membrane (Hybond, GE Healthcare, Chalfont St. Giles, UK). Before immunodetection, membranes were blocked with 5% (w/v) BSA in a 0.1% Tween-PBS solution. Membranes were then incubated with primary antibodies overnight at 4° C. with agitation followed by incubation with a goat anti-rabbit or anti-mouse IgGs coupled to IRDye800 (LI-COR Biosciences, Lincoln, Nebr.). Immunodetection was then performed using an infrared imager (Odyssey Imager, LI-COR Biosciences). Relative protein expression levels were calculated using the ImageJ software.

### Example IV

### DNA Methylation Analysis

DNA was purified from 14 pairs of tumoral and non-tumoral prostate biopsies and from DU145 and LNCaP cells treated with 5axa-dC using DNeasy Blood & Tissue Kit (Qiagen, #69504). Concentration, yield and purity of gDNA samples were measured using spectrometry. All samples provided good gDNA yield and quality (A260/A280 ratio between 1.7 and 2.0). Gold standard pyrosequencing technology was used to determine base-specific cytosine methylation levels located upstream of guanines (sequence called CpG dinucleotides). Three potential CTCF binding sites were targeted close to exon 25 and alternative exon 25 of the PCSK6 gene identified by transcription factor ChIP-seq from ENCODE project with Factorbook (UCSC Genome Bioinformatics) (FIGS. 4A and B). Pyrosequencing assays combine sodium bisulfite DNA conversion chemistry gDNA (600 ng; EpiTech Bisulfite Kits; Qiagen, #59104), polymerase chain reaction (PCR) amplification of Na-Bis treated DNA (Pyromark PCR Kit; Qiagen, #978703) and sequencing by synthesis assay of the PCR products (Pyromark Gold Q24 Reagents; Qiagen, #978802), as previously described (Guay et al., 2012, Epigenetics, 7: 464-472). Briefly, sodium bisulfite preferentially deaminates unmethylated cytosines to thymines (after PCR amplification), whereas methyl-cytosines remain unmodified. During pyrosequencing analysis, dTTPs and dCTPs are added sequentially at each CpG site and the relative peak height of dTTP versus dCTP obtained on pyrograms allowed to determine base-specific DNA methylation levels (PyroMark Q24 1.1.10). PCR and sequencing primers were designed using PyroMark Assay Design software v.2.0.1.15. Overall, nine potentially methylated cytosines (in a CpG dinucleotides context) were analyzed at the PCSK6 gene locus.

### Example V

### Chromatin Immunoprecipitation

Cells were cultured in 150 mm culture dishes and submitted to crosslinking through the addition of formaldehyde to a final concentration of 1.1% for 10 min at room temperature followed by a quenching step with 125 mM glycine for another 5 min. Cells were washed twice with ice-cold PBS, collected using a cell scraper and frozen in liquid nitrogen and stored at −80° C. until analyses. Cell pellets were resuspended in HEPES 10 mM pH 6.5; 0.5 mM EDTA, 0.25% Triton-X-100 and centrifugated at 4,000 rpm at 4° C. for 5 min. Pellet was further lysed by adding 200 μL of Tris 50 mM pH 8.1; 10 mM EDTA, 1% SDS and passing 3 times through a 28 G syringe. Cell preparations were incubated 1 h at 4° C. with constant agitation and nuclei were pelleted by a centrifugation at 5,000 rpm. 400 μL of water was added (to dilute EDTA) to the nuclei and the solution was sonicated 3×10 sec at intensity 6/10 on ice followed by a 13,000 rpm centrifugation for 10 min at 4° C. 1.1×10⁴ micrococal nuclease gel units (New England Biolabs) were added with its manufactured buffers and BSA and incubated for 5 min on ice before being neutralized with 0.5 M EDTA (final concentration: 10 mM). DNA fragmentation was routinely assessed by agarose gel electrophoresis using 10 μL of the fragmented DNA solution (beforehand treated with RNase A 10 μg/mL for 10 min). For each IP, 350 μg of DNA were used (based on concentration determined using the OD_{260 nM}) and 5% of the corresponding volumes were kept aside as Input DNA for quantitation. Each IP was completed to 1 mL with IP buffer (Tris 16.7 mM pH 8.1; 167 mM NaCl, 1.2 mM EDTA, 1.1% Triton X-100, 0.01% SDS). Immunoprecipitations were carried out by incubating the DNA with 10 μg of specific antibody or normal IgG overnight at 4° C. Antibody-DNA complex were retrieved by adding 40 μL of Protein A MagneResyn beforehand incubated 1 h at 4° C. with 350 μg/mL of salmon sperm DNA. After 1 h with the beads, beads were sequentially washed 5 min twice with each of the following buffers: i) Tris 20 mM pH 8.1; 150 mM NaCl, 2 mM EDTA, 1% Triton-X-100, 0.1% SDS, ii) Tris 20 mM pH 8.1; 500 mM NaCl, 2 mM EDTA, 1% Triton-X-100, 0.1% SDS, iii) Tris 10 mM pH 8.1; 1 mM EDTA, 1% NP40, 1% Na-deoxycholate, 0.25 M LiCl and iv) Tris 10 mM pH 8.1; 0.1 mM EDTA. Protease inhibitor (Complete Mini; Roche) was added to all buffers immediately before use Beads (and their corresponding 5% Input DNA) were finally resuspended in 0.1 M NaHCO₃; 1% SDS and heated at 65° C. with frequent agitation to elute DNA from the beads. Following centrifugation, the supernatant containing the DNA were incubated with 50 μg/mL proteinase K and 25 μg/mL RNase A overnight at 65° C. DNA was finally purified using QIAquick PCR purification Kit following manufacturer guidelines and 3 M sodium acetate to adjust the solution pH. ChIP isolated DNA was eluted in 50 μL of TE buffer whereas the corresponding 5% input DNA was eluted in 250 μL to put the concentration at 1% equivalent. 3 μL of the purified DNA were further used for qPCR analyses and quantitation was established using the 1% input as a standard.

### Example VI

### Normal RNA and Tumor cDNA Analysis

Normal RNA standardized preparations were obtained from Clontech Laboratories (Total RNA Master Panel II; Mountain View, Calif.). These consist of total RNA from controlled origins controlled by capillary electrophoresis and denaturing formaldehyde agarose gel electrophoresis. 1 μg of RNA was used for further RT-qPCR analyzes. For normal and tumoral cDNA, cDNA were obtained from Origene (Rockville, Md.) Cancer Survey cDNA Array covering different cancers across identical qPCR plates. All samples were analyzed by the addition of premixed SYBRgreen and primers for a single transcript per plate and using the supplied actin primers as normalizer gene according to the manufacturer's instructions.

### Example VII

### Stable Isotope Labelling by Amino Acids in Cell Culture and Secretome Preparation

For SILAC labelling, cells to be labelled were resurrected from their cryovials in RPMI 1640 without L-arginine and L-lysine (ThermoFisher Scientific) complemented with 42 mg/L [¹³C₆]-L-Arginine, 73 mg/L [¹³C₆]-L-Lysine (Cambridge Isotope Laboratories, Inc, MA) and dialyzed fetal bovine serum. After at least three passages in heavy medium, cells were checked for complete labelling using the method described in Scmidt et al. ((2007, Rapid Commun Mass Spectrom, 21: 3919-3926) and cryopreserved for further uses. Proline conversion was manually assessed in Pro containing peptides and was found to be <0.1%. For conditioned media productions, fixed cell numbers were plated in p150 mm culture plates (4.5×10⁶ for DU145 and 6×10⁶ for LNCaP) for 36 h before washing the cells and adding fresh serum-free medium. Cells were allowed to conditions the medium for 24 h, after what it was collected, centrifuged for 5 min at 1,000×g, filtered on a 0.22 μm syringe unit and flash frozen in liquid nitrogen until use. Upon thawing, protease inhibitor cocktail was added (final concentration 1×, Mini protease inhibitor with EDTA, Roche) and concentrated on a 3 kDa Amicon-Ultra centrifugal unit (Millipore), typically 15 mL were concentrated to 1.5 mL before being pooler 1:1 (volume:volume) with the non-labelled conditions (ex. Non-Target: shPACE4) precipitated by the addition of 9 volumes of acetone:methanol (8:1, MS grade Fisher reagents). Precipitation was performed overnight at −80° C. Protein precipitates were collected by centrifugation at 17.000× g, 30 min at 4° C., washed three times with methanol by inversion (4° C., 10 min). Washed pellets were then solubilized in SAGE-Elf sample loading solution (with SDS) and heated at 95° C. for 10 min with DTT (final concentration 10 mM). Samples were loaded on SAGE gel cassettes (3% agarose) and migrated for 1 h before being electro-eluted into 13 fractions. Fractions were collected, diluted 5 times and used for DTT reduction (5 mM, room temperature, 30 min), iodoacetamie alkylation (5 mM, room temperature, 30 min dark) and quenching with DTT (5 mM, room temperature, 30 min, dark). Protein were then digested with trypsin 1 μg per 100 μg (determined using BCA protein titration assay) overnight at 37° C. in a thermo-shaker. Peptide solutions were acidified with 5 μL formic acid before adding 1 volume of KCl 4M. Samples were vortexed 1 min and allowed to stand for 10 min at room temperature before being submitted to ethyl-acetate organic liquid-liquid extraction by adding the maximal volume of ethyl-acetate in the tube. Organic phase was discarded and the aqueous phase containing the peptides was resubmitted to the extraction twice to ensure complete SDS removal. Residual organics were evaporated by letting the tube stand open in a chemical hood for 20 min. Peptide solutions were then re-acidified by adding 5 μL formic acid and peptide were cleaned by solid-phase extraction (Strata-X 33u polymeric reversed phase, 30 mg/l mL) using the following procedure on a vacuum manifold (each solution was allowed to completely drain before adding the next one): 1 mL ACN, 1 mL H₂O 0.1% formic acid, acidified peptide solution, 1 mL H₂O 0.1% formic acid, 50% ACN 0.1% formic acid: for elution). Peptide were further dried in a Speed-Vac system and restituted in H₂O 0.2% formic acid, 3% DMSO and submitted to LC-MS/MS analysis.

Acquisition was performed with a Sciex TripleTOF 5600 (Sciex, Foster City, Calif., USA) equipped with an electrospray interface with a 25 μm iD capillary and coupled to an Eksigent pUHPLC (Eksigent, Redwood City, Calif., USA). Analyst TF 1.6 software was used to control the instrument and for data processing and acquisition. The source voltage was set to 5.2 kV and maintained at 325° C., curtain gas was set at 27 psi, gas one at 12 psi and gas two at 10 psi. Acquisition was performed in Information Dependant Acquisition (IDA). Separation was performed on a reversed phase HALO C₁₈-ES column 0.3 μm i.d., 2.7 μm particles, 150 mm long (Advance Materials Technology, Wilmington, Del.) which was maintained at 60° C. Samples were injected by loop overfilling into a 5 μL loop. For the 120 minute LC gradient, the mobile phase consisted of the following solvent A (0.2% v/v formic acid and 3% DMSO v/v in water) and solvent B (0.2% v/v formic acid and 3% DMSO in ethanol) at a flow rate of 3 μL/min.

The gradient was as follows: 0-88 minutes from 2% B to 30% B, 88-108 minutes from 30% B to 55% B, 108-115 minutes from 55% B to 95% B, hold 95% B for 5 minutes followed by a 1 minute post flush at final conditions. The raw data was processed by the Protein Pilot software (Sciex, Foster City, Calif., USA). Following peptide and protein identification, an unlabelled:labelled (light:heavy) ratio as well as a p-Value was calculated by the software from the individual peptides for every protein. From the obtained protein identifications, secreted proteins were retrieved using ProteINSIDE (Kaspric et al., 2015), only proteins predicted to be secreted (based on the presence of a signal peptide) were considered. Only L/H ratio <1 were considered since many proteins detected with L/H>1 were composed of peptides with high homology with bovine proteins which may lead to misinterpretation as residual albumin was present in the conditioned medium samples. For proteins only identified in the heavy condition (L/H=0), a primary exclusion criterion based on P-value was applied, only P<0.05 were preserved. All proteins were manually searched for PC-based or PC-like processing events, using both Uniprot PTM/Processing data or by ProP 1.0 Server.

For IP-MS analysis, each condition was injected twice. First, acquisition was performed in Information Dependant Acquisition for the generation of the ion library. The samples were then reinjected in and acquired with variable size windows in SWATH mode for the quantification. Separation was performed on a reversed phase HALO C₁₈-ES column 0.3 μm i.d., 2.7 μm particles, 150 mm long (Advance Materials Technology, Wilmington, Del.) which was maintained at 60° C. Samples were injected by loop overfilling into a 5 μL loop. For the 60 minute LC gradient, the mobile phase consisted of the following solvent A (0.2% v/v formic acid and 3% DMSO v/v in water) and solvent B (0.2% v/v formic acid and 3% DMSO in ethanol) at a flow rate of 3 μL/min. The gradient was as follows: 0-44 minutes from 2% B to 30% B, 44-54 minutes from 30% B to 55% B, 54-57 minutes from 55% B to 95% B, hold 95% B for 5 minutes followed by a 5 minute post flush at final conditions. The protein database and the ion library were generated by analysing simultaneously every IDA files with the ProteinPilot software (Sciex, Foster City, Calif., USA). This database was then used to quantify the proteins with the SWATH quantification tool in the Peakview software (Sciex, Foster City, Calif., USA). Peakview outputs an area under the curve of the chromatograms for each peptides that was detected in the sample, as well as a peak score and a false discovery rate. A peptide was considered as correctly integrated if the peak score was higher than 0.5 or if the false discovery rate was lower than 1%. Protein quantification represents the sum of every correctly integrated peptides. To correct the differences in the amount of peptides that was loaded on the column, every protein was divided by a correction factor that took into account the total protein amount of a sample compared to the average of the total protein amount of all the samples.

### Example VIII

### Peptide Synthesis

ML peptide and its derivatives (Peg8-ML and C23) were synthesized as previously described in Kwiatkowska et al. (2014, Journal of Medicinal Chemistry, 57: 98-109). The synthesis GDF-15 spanning peptide was performed manually by a standard solid-phase peptide method on TentaGel S RAM-amide resin (0.5 g, 0.13 mmol/g). Briefly, Fmoc deprotection was carried out with 20% piperidine in DMF (5 and 10 minutes), Fmoc-protected amino acids (3 equiv), O-(7-azabenzotriazol-1-yl)-N,N,N0,N0-tetramethyluronium hexafluorophosphate (HATU, 3 equiv), 1-hydroxy-6-chloro-benzotriazole (6-CI-HOBt, 3 equiv) and N,N-diisopropylethylamine (DIPEA, 9 equiv) were used for coupling. Completion of the reaction was confirmed by the Kaiser test. After final Fmoc deprotection GDF-15 peptide having a L-Gln residue at its N-terminus was acetylated to prevent formation of pyroglutamate using the mixture acetic anhydride/DIPEA/dichloromethane (15:15:70 v/v/v, 10 ml). Peptide was cleaved from the resin using a cocktail of trifluoroacetic acid (TFA)/H₂O/triisopropylsilane (TIS) (95:2.5:2.5 v/v/v, 20 ml) for 3 h at room temperature. The products were precipitated in cold diethyl ether, collected by centrifugation, dissolved and freeze-dried to a white solid. The crude peptides were purified by preparative HPLC (VARIAN ProStar). The fractions containing pure product were pooled and lyophylized. The identity and purity of peptides (97%) was confirmed by HRMS (TripleTOF 5600, ABSciex) and analytical HPLC (Agilent Technologies 1100 system) equipped with a diode array detector with Agilent Eclipse XDB C₁₈ column.

GDF-15 peptide (40 μg) was incubated at 37° C. with recombinant PACE4 or soluble furin (16U) in 100 mM Hepes buffer containing 1 mM CaCl₂, 1 mM 3-mercaptoethanol, and 1.8 mg/mL BSA, pH 7.5 (total sample volume: 300 μl) over a period of 1 h. Following controls were used: buffer alone and a peptide or an enzyme incubated in buffer. After incubation, the reactions were immediately analyzed by the analytical HPLC (Agilent Technologies, 1100 series with a diode array detector and a fraction collector; injection volume: 95 μl, gradient: 2 to 25% [A] in [B] in 50 min; [A]0.1% aq TFA and [B] acetonitrile+0.1% aq TFA; column: an Agilent Eclipse XDB C18 column (5 μm, 4.6×250 mm). The collected fractions were analysed by SELDI-TOF mass spectrometer (Bio-Rad Laboratories) to identify the cleavage product.

### Example IX

### Plasma Collection

Blood samples were drawn just prior to the prostatectomy procedure from patients who had agreed to participate. For normal patients, samples were collected from patients referred for a PSA titration who had agreed to participate and signed a consent form. Blood was collected in EDTA-coated tubes (Vacutainer; BD) and centrifuged for 15 min at 5,000×g (4° C.). Plasma was then aliquoted and stored at −80° C. until use for ELISA assay (described in the detailed methods section).

While the disclosure has been described in connection with specific embodiments thereof, it will be understood that it is capable of further modifications and this application is intended to cover any variations, uses, or adaptations, including such departures from the present disclosure as come within known or customary practice within the art, and as may be applied to the essential features hereinbefore set forth, and as follows in the scope of the appended claims.

1: A method for detecting a cancer in a subject comprising the steps of:
a) obtaining a biological sample from the subject;
b) contacting an analyte specific reagent specifically binding to PACE4-altCT with the biological sample under conditions so as to allow the formation of an analyte-PACE4-altCT complex; and
c) detecting said cancer by detecting the analyte-PACE4-altCT complex in said biological sample.

2-3. (canceled)

4: The method of claim 1, further comprising the step of detecting the presence of PACE4-FL in said biological sample and calculating a ratio of PACE4-altCT/PACE4-FL wherein a ration of above 2 is indicative of the presence of said cancer.

5: The method of claim 1, wherein the sample is a blood sample, urine, a tissue specimen, a biopsy needle washes, or circulating cells.

6: The method of claim 1, wherein the analyte is an antibody, a peptide, a primer or a probe.

7-8. (canceled)

9: The method of claim 6, wherein said antibody specifically binds to SEQ ID NO: 9.

10: The method of claim 6, wherein said antibody specifically binds to an epitope comprising the amino acid sequence set forth in any one of SEQ ID NOs: 18, 23, 24, 25, and 26.

11. (canceled)

12: The method of claim 6, wherein the probe is an oligonucleotide or a siRNA molecule.

13: The method of claim 6, wherein said probe specifically binds to a nucleotide sequence comprising SEQ ID NOs: 5 or 6.

14: The method of claim 12, wherein said siRNA comprises the nucleotide sequence set forth in SEQ ID NOs: 10, 11, 12, 13, 14, 15 or 16.

15-18. (canceled)

19: The method of claim 1, wherein said cancer is in at least one of lungs, thyroid, adrenals, testis, endometrium, pancreas, oesophagus, prostate, ovary, liver, breast, colon, stomach, kidney, bladder, brain, cervix, and lymphoid tissues.

20: The method of claim 19, wherein said cancer is a prostate cancer.

21-35. (canceled)

36: An antibody specifically binding to PACE4-altCT.

37-38. (canceled)

39: The antibody of claim 36, wherein said antibody specifically binds to SEQ ID NO: 9.

40: The antibody of claim 36, wherein said antibody specifically binds to an epitope comprising the amino acid sequence set forth in any one of SEQ ID NOs: 18, 23, 24, 25, and 26.

41: The antibody of claim 36, for detecting and/or treating cancer.

42: A method of treating a cancer in a patient comprising administering an inhibitor of PACE4-altCT to a patient in need thereof.

43-79. (canceled)

80: The method of claim 42, wherein said inhibitor of PACE4-altCT is an antibody as defined in claim 36.

81: The method of claim 42, wherein said inhibitor of PACE4-altCT is a probe as defined in claim 13 or an siRNA as defined in claim 14.

82: The method of claim 42, wherein said cancer is in at least one of lungs, thyroid, adrenals, testis, endometrium, pancreas, oesophagus, prostate, ovary, liver, breast, colon, stomach, kidney, bladder, brain, cervix, and lymphoid tissues.

83: The method of claim 42, wherein said cancer is a prostate cancer.


SEQUENCE LISTING (42 sequences)

SEQ ID NO: 1 - protein, 969 aa, Human
  PACE4
MPPRAPPAPGPRPPPRAAAATDTAAGAGGAGGAGGAGGPGFRPLAPRPWRWLLLLALPAACSAPPPRPVYTNHWAVQVLGGPAEADRVAAAHGYLNLGQIGNLEDYYHFYHSKTFKRSTLSSRGPHTFLRMDPQVKWLQQQEVKRRVKRQVRSDPQALYFNDPIWSNMWYLHCGDKNSRCRSEMNVQAAWKRGYTGKNVVVTILDDGIERNHPDLAPNYDSYASYDVNGNDYDPSPRYDASNENKHGTRCAGEVAASANNSYCIVGIAYNAKIGGIRMLDGDVTDVVEAKSLGIRPNYIDIYSASWGPDDDGKTVDGPGRLAKQAFEYGIKKGRQGLGSIFVWASGNGGREGDYCSCDGYTNSIYTISVSSATENGYKPWYLEECASTLATTYSSGAFYERKIVTTDLRQRCTDGHTGTSVSAPMVAGIIALALEANSQLTWRDVQHLLVKTSRPAHLKASDWKVNGAGHKVSHFYGFGLVDAEALVVEAKKWTAVPSQHMCVAASDKRPRSIPLVQVLRTTALTSACAEHSDQRVVYLEHVVVRTSISHPRRGDLQIYLVSPSGTKSQLLAKRLLDLSNEGFTNWEFMTVHCWGEKAEGQWTLEIQDLPSQVRNPEKQGKLKEWSLILYGTAEHPYHTFSAHQSRSRMLELSAPELEPPKAALSPSQVEVPEDEEDYTAQSTPGSANILQTSVCHPECGDKGCDGPNADQCLNCVHFSLGSVKTSRKCVSVCPLGYFGDTAARRCRRCHKGCETCSSRAATQCLSCRRGFYHHQEMNTCVTLCPAGFYADESQKNCLKCHPSCKKCVDEPEKCTVCKEGFSLARGSCIPDCEPGTYFDSELIRCGECHHTCGTCVGPGREECIHCAKNFHFHDWKCVPACGEGFYPEEMPGLPHKVCRRCDENCLSCAGSSRNCSRCKTGFTQLGTSCITNHTCSNADETFCEMVKSNRLCERKLFIQFCCRTCLLAG

SEQ ID NO: 2 - DNA, 4403 bp, Human
  PACE4
cgggaacgcgccgcggccgcctcctcctccccggctcccgcccgcggcggtgttggcggcggcggtggcggcggcggcggcgcttccccggcgcggagcggctttaaaaggcggcactccaccccccggcgcactcgcagctcgggcgccgcgcgagcctgtcgccgctatgcctccgcgcgcgccgcctgcgcccgggccccggccgccgccccgggccgccgccgccaccgacaccgccgcgggcgcggggggcgcggggggcgcggggggcgccggcgggcccgggttccggccgctcgcgccgcgtccctggcgctggctgctgctgctggcgctgcctgccgcctgctccgcgcccccgccgcgccccgtctacaccaaccactgggcggtgcaagtgctgggcggcccggccgaggcggaccgcgtggcggcggcgcacggctacctcaacttgggccagattggaaacctggaagattactaccatttttatcacagcaaaacctttaaaagatcaaccttgagtagcagaggccctcacaccttcctcagaatggacccccaggtgaaatggctccagcaacaggaagtgaaacgaagggtgaagagacaggtgcgaagtgacccgcaggccctttacttcaacgaccccatttggtccaacatgtggtacctgcattgtggcgacaagaacagtcgctgccggtcggaaatgaatgtccaggcagcgtggaagaggggctacacaggaaaaaacgtggtggtcaccatccttgatgatggcatagagagaaatcaccctgacctggccccaaattatgattcctacgccagctacgacgtgaacggcaatgattatgacccatctccacgatatgatgccagcaatgaaaataaacacggcactcgttgtgcgggagaagttgctgcttcagcaaacaattcctactgcatcgtgggcatagcgtacaatgccaaaataggaggcatccgcatgctggacggcgatgtcacagatgtggtcgaggcaaagtcgctgggcatcagacccaactacatcgacatttacagtgccagctgggggccggacgacgacggcaagacggtggacgggcccggccgactggctaagcaggctttcgagtatggcattaaaaagggccggcagggcctgggctccattttcgtctgggcatctgggaatggcgggagagagggggactactgctcgtgcgatggctacaccaacagcatctacaccatctccgtcagcagcgccaccgagaatggctacaagccctggtacctggaagagtgtgcctccaccctggccaccacctacagcagtggggccttttatgagcgaaaaatcgtcaccacggatctgcgtcagcgctgtaccgatggccacactgggacctcagtctctgcccccatggtggcgggcatcatcgccttggctctagaagcaaacagccagttaacctggagggacgtccagcacctgctagtgaagacatcccggccggcccacctgaaagcgagcgactggaaagtaaacggcgcgggtcataaagttagccatttctatggatttggtttggtggacgcagaagctctcgttgtggaggcaaagaagtggacagcagtgccatcgcagcacatgtgtgtggccgcctcggacaagagacccaggagcatccccttagtgcaggtgctgcggactacggccctgaccagcgcctgcgcggagcactcggaccagcgggtggtctacttggagcacgtggtggttcgcacctccatctcacacccacgccgaggagacctccagatctacctggtttctccctcgggaaccaagtctcaacttttggcaaagaggttgctggatctttccaatgaagggtttacaaactgggaattcatgactgtccactgctggggagaaaaggctgaagggcagtggaccttggaaatccaagatctgccatcccaggtccgcaacccggagaagcaagggaagttgaaagaatggagcctcatactgtatggcacagcagagcacccgtaccacaccttcagtgcccatcagtcccgctcgcggatgctggagctctcagccccagagctggagccacccaaggctgccctgtcaccctcccaggtggaagttcctgaagatgaggaagattacacagctcaatccaccccaggctctgctaatattttacagaccagtgtgtgccatccggagtgtggtgacaaaggctgtgatggccccaatgcagaccagtgcttgaactgcgtccacttcagcctggggagtgtcaagaccagcaggaagtgcgtgagtgtgtgccccttgggctactttggggacacagcagcaagacgctgtcgccggtgccacaaggggtgtgagacctgctccagcagagctgcgacgcagtgcctgtcttgccgccgcgggttctatcaccaccaggagatgaacacctgtgtgaccctctgtcctgcaggattttatgctgatgaaagtcagaaaaattgccttaaatgccacccaagctgtaaaaagtgcgtggatgaacctgagaaatgtactgtctgtaaagaaggattcagccttgcacggggcagctgcattcctgactgtgagccaggcacctactttgactcagagctgatcagatgtggggaatgccatcacacctgcggaacctgcgtggggccaggcagagaagagtgcattcactgtgcgaaaaacttccacttccacgactggaagtgtgtgccagcctgtggtgagggcttctacccagaagagatgccgggcttgccccacaaagtgtgtcgaaggtgtgacgagaactgcttgagctgtgcaggctccagcaggaactgtagcaggtgtaagacgggcttcacacagctggggacctcctgcatcaccaaccacacgtgcagcaacgctgacgagacattctgcgagatggtgaagtccaaccggctgtgcgaacggaagctcttcattcagttctgctgccgcacgtgcctcctggccgggtaagggtgcctagctgcccacagagggcaggcactcccatccatccatccgtccaccttcctccagactgtcggccagagtctgtttcaggagcggcgccctgcacctgacagctttatctccccaggagcagcatctctgagcacccaagccaggtgggtggtggctcttaaggaggtgttcctaaaatggtgatatcctctcaaatgctgcttgttggctccagtcttccgacaaactaacaggaacaaaatgaattctgggaatccacagctctggctttggagcagcttctgggaccataagtttactgaatcttcaagaccaaagcagaaaagaaaggcgcttggcatcacacatcactcttctccccgtgcttttctgcggctgtgtagtaaatctccccggcccagctggcgaaccctgggccatcctcacatgtgacaaagggccagcagtctacctgctcgttgcctgccactgagcagtctggggacggtttggtcagactataaataagataggtttgagggcataaaatgtatgaccactggggccggagtatctatttctacatagtcagctacttctgaaactgcagcagtggcttagaaagtccaattccaaagccagaccagaagattctatcccccgcagcgctctcctttgagcaagccgagctctccttgttaccgtgttctgtctgtgtcttcaggagtctcatggcctgaacgaccacctcgacctgatgcagagccttctgaggagaggcaacaggaggcattctgtggccagccaaaaggtaccccgatggccaagcaattcctctgaacaaaatgtaaagccagccatgcattgttaatcatccatcacttcccattttatggaattgcttttaaaatacatttggcctctgcccttcagaagactcgtttttaaggtggaaactcctgtgtctgtgtatattacaagcctacatgacacagttggatttattctgccaaacctgtgtaggcattttataagctacatgttctaatttttaccgatgttaattattttgacaaatatttcatatattttcattgaaatgcacagatctgcttgatcaattcccttgaatagggaagtaacatttgccttaaattttttcgacctcgtctttctccatattgtcctgctcccctgtttgacgacagtgcatttgccttgtcacctgtgagctggagagaacccagatgttgtttattgaatctacaactctgaaagagaaatcaatgaagcaagtacaatgttaaccctaaattaataaaagagttaacatccc

SEQ ID NO: 3 - DNA, 98 bp, Human
  Exon 25 cons
ctgacgagacattctgcgagatggtgaagtccaaccggctgtgcgaacggaagctcttcattcagttctgctgccgcacgtgcctcctggccgggtaa

SEQ ID NO: 4 - DNA, 1335 bp, Human
  3′UTR cons
gggtgcctagctgcccacagagggcaggcactcccatccatccatccgtccaccttcctccagactgtcggccagagtctgtttcaggagcggcgccctgcacctgacagctttatctccccaggagcagcatctctgagcacccaagccaggtgggtggtggctcttaaggaggtgttcctaaaatggtgatatcctctcaaatgctgcttgttggctccagtcttccgacaaactaacaggaacaaaatgaattctgggaatccacagctctggctttggagcagcttctgggaccataagtttactgaatcttcaagaccaaagcagaaaagaaaggcgcttggcatcacacatcactcttctccccgtgcttttctgcggctgtgtagtaaatctccccggcccagctggcgaaccctgggccatcctcacatgtgacaaagggccagcagtctacctgctcgttgcctgccactgagcagtctggggacggtttggtcagactataaataagataggtttgagggcataaaatgtatgaccactggggccggagtatctatttctacatagtcagctacttctgaaactgcagcagtggcttagaaagtccaattccaaagccagaccagaagattctatcccccgcagcgctctcctttgagcaagccgagctctccttgttaccgtgttctgtctgtgtcttcaggagtctcatggcctgaacgaccacctcgacctgatgcagagccttctgaggagaggcaacaggaggcattctgtggccagccaaaaggtaccccgatggccaagcaattcctctgaacaaaatgtaaagccagccatgcattgttaatcatccatcacttcccattttatggaattgcttttaaaatacatttggcctctgcccttcagaagactcgtttttaaggtggaaactcctgtgtctgtgtatattacaagcctacatgacacagttggatttattctgccaaacctgtgtaggcattttataagctacatgttctaatttttaccgatgttaattattttgacaaatatttcatatattttcattgaaatgcacagatctgcttgatcaattcccttgaatagggaagtaacatttgccttaaattttttcgacctcgtctttctccatattgtcctgctcccctgtttgacgacagtgcatttgccttgtcacctgtgagctggagagaacccagatgttgtttattgaatctacaactctgaaagagaaatcaatgaagcaagtacaatgttaaccctaaattaataaaagagttaacatcccatggcaaaaaa

SEQ ID NO: 5 - DNA, 92 bp, Human
  Exon 25 ALT
gggacatttggcaacgtctggagacattttgggttgtcacaactggaaggatgtactcacatccagtgggtggaggccaggaatgctgctaa

SEQ ID NO: 6 - DNA, 164 bp, Human
  3′UTR alt
gcaccctgcaatgcacagcagcccccatgacaaagaactgtccagaccaaaatgtcaatagtgccgaggttgagaaattccggccaaaagaaatggaatcaaaaaagaaaattgtttgcaacttcatagagataccctcaatgaataaaaccacatatggtttg

SEQ ID NO: 7 - protein, 7 aa, Human
  end of Exon 24 (cons)
TNHTCSN

SEQ ID NO: 8 - protein, 32 aa, Human
  Exon 25 cons
ADETFCEMVKSNRLCERKLFIQFCCRTCLLAG

SEQ ID NO: 9 - protein, 30 aa, Human
  Exon 25 alt
GDIWQRLETFWVVTTGRMYSHPVGGGQECC

SEQ ID NO: 10 - DNA, 19 bp, Artificial Sequence
  siRNA for PACE4-FL (cons)
ctcttaaggaggtgttcct

SEQ ID NO: 11 - DNA, 19 bp, Artificial Sequence
  siRNA for PACE4-FL (cons)
gaccataagtttactgaat

SEQ ID NO: 12 - DNA, 19 bp, Artificial Sequence
  siRNA for PACE4-FL (cons)
gtatctatttctacatagt

SEQ ID NO: 13 - DNA, 19 bp, Artificial Sequence
  siRNA for PACE4-FL (cons)
caagcaattcctctgaaca

SEQ ID NO: 14 - DNA, 19 bp, Artificial Sequence
  siRNA for PACE4-FL (cons)
gacaaatatttcatatatt

SEQ ID NO: 15 - DNA, 19 bp, Artificial Sequence
  siRNA for PACE4-altCT (alt)
ggaaggatgtactcacatc

SEQ ID NO: 16 - DNA, 19 bp, Artificial Sequence
  siRNA for PACE4-altCT (alt)
gatgtactcacatccagtg

SEQ ID NO: 17 - protein, 27 aa, Artificial Sequence
  polyclonal antibody epitope- Exon 25 cons
CEMVKSNRLCERKLFIQFCCRTCLLAG

SEQ ID NO: 18 - protein, 15 aa, Artificial Sequence
  polyclonal antibody epitope- Exon 25 alt
TGRMYSHPVGGGQEC

SEQ ID NO: 19 - protein, 8 aa, Artificial Sequence
  monoclonal antibody epitope- Exon 25 cons1-2-3
EMVKSNRL

SEQ ID NO: 20 - protein, 10 aa, Artificial Sequence
  monoclonal antibody epitope- Exon 25 cons4-5-6
SNRLCERKLF

SEQ ID NO: 21 - protein, 8 aa, Artificial Sequence
  monoclonal antibody epitope- Exon 25 cons7-8-9
ERKLFIQF

SEQ ID NO: 22 - protein, 8 aa, Artificial Sequence
  monoclonal antibody epitope- Exon 25 cons10-11-12
CRTCLLAG

SEQ ID NO: 23 - protein, 12 aa, Artificial Sequence
  monoclonal antibody epitope- Exon 25 alt1
WVVTTFRMYSHP

SEQ ID NO: 24 - protein, 10 aa, Artificial Sequence
  monoclonal antibody epitope- Exon 25 alt2-3-4
TTFRMYSHPV

SEQ ID NO: 25 - protein, 10 aa, Artificial Sequence
  monoclonal antibody epitope- Exon 25 alt5-6-7
RMYSHPVGGG

SEQ ID NO: 26 - protein, 10 aa, Artificial Sequence
  monoclonal antibody epitope- Exon 25 alt8-9-10
YSHPVGGGQE

SEQ ID NO: 27 - protein, 12 aa, Artificial Sequence
  monoclonal antibody epitope- p1
PSQVRNPEKQGK

SEQ ID NO: 28 - protein, 12 aa, Artificial Sequence
  monoclonal antibody epitope- p2-3-4
DKRPRSIPLVQV

SEQ ID NO: 29 - protein, 12 aa, Artificial Sequence
  monoclonal antibody epitope- p5-6-7
PELEPPKAALSP

SEQ ID NO: 30 - protein, 12 aa, Artificial Sequence
  monoclonal antibody epitope- p8-9-10
SRPAHLKASDWK

SEQ ID NO: 31 - protein, 12 aa, Artificial Sequence
  monoclonal antibody epitope- p11
TDGHTGTSVSAP

SEQ ID NO: 32 - protein, 12 aa, Artificial Sequence
  monoclonal antibody epitope- p12-13-14
ERNHPDLAPNYD

SEQ ID NO: 33 - protein, 12 aa, Artificial Sequence
  monoclonal antibody epitope- p15
PAGFYADESQKN

SEQ ID NO: 34 - protein, 12 aa, Artificial Sequence
  monoclonal antibody epitope- pro1
GYLNLGQIGNLE

SEQ ID NO: 35 - protein, 12 aa, Artificial Sequence
  monoclonal antibody epitope- pro2
QIGNLEDYYHFY

SEQ ID NO: 36 - protein, 12 aa, Artificial Sequence
  monoclonal antibody epitope- pro3
NLEDYYHFYHSK

SEQ ID NO: 37 - protein, 12 aa, Artificial Sequence
  monoclonal antibody epitope- pro4-5
RSTLSSRGPHTF

SEQ ID NO: 38 - protein, 12 aa, Artificial Sequence
  monoclonal antibody epitope- pro6
HTFLRMDPQVKW

SEQ ID NO: 39 - protein, 12 aa, Artificial Sequence
  monoclonal antibody epitope- pro7-8-9
QVLGGPAEADRV

SEQ ID NO: 40 - DNA, 38 bp, Artificial Sequence
  cDNA cloning primer
ggccacgcgtcgactagtactttttttttttttttttv

SEQ ID NO: 41 - DNA, 18 bp, Artificial Sequence
  gene specific primer
acccagaagagatgccgg

SEQ ID NO: 42 - DNA, 20 bp, Artificial Sequence
  3′RACE primer
ggccacgcgtcgactagtac